SEQUENCE LISTING (25 sequences)

SEQ ID NO: 1 - DNA, 2717 bp, synthetic construct
  misc_feature 1..2717: note = engineered anti-HER antibody
  misc_feature 1..60: note = misc - IL2 signal/leader peptide
  misc_feature 439..714: note = Misc - CH1
  misc_feature 715..1410: note = Misc - CH1
  misc_feature 1422..2009: note = IRES
  misc_feature 2010..2069: note = Misc - IL2 signal/leader peptide
  misc_feature 2070..2391: note = Misc - variable light
  misc_feature 2407..2711: note = Misc - light chain constant region
  source 1..2717: mol_type = other DNA
  CDS 61..423: note = heavy variable
atgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggcaagtgataaggccggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgtaccgcatgcaactcctgtcttgcattgcactaagtcttgcacttgtcacaaacagtgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctgataa

SEQ ID NO: 2 - protein, 121 aa, synthetic construct
  REGION 1..121: note = Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSA

SEQ ID NO: 3 - protein, 470 aa, synthetic construct
  REGION 1..470: note = engineered anti-HER heavy chain
  REGION 1..20: note = MISC_FEATURE - heavy chain signal peptide
  REGION 21..140: note = MISC_FEATURE - heavy chain variable region
  REGION 141..259: note = MISC_FEATURE - heavy chain constant region 1
  REGION 260..470: note = MISC_FEATURE - heavy chain FC
MYRMQLLSCIALSLALVTNSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
  REGION 1..214: note = Engineered anti-Her2 light chain
  REGION 1..20: note = MISC_FEATURE - signal sequence
  REGION 21..130: note = MISC_FEATURE - light chain variable region
  REGION 131..214: note = MISC_FEATURE - light chain constant region
MYRMQLLSCIALSLALVTNSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYAC

SEQ ID NO: 5 - DNA, 6694 bp, synthetic construct
  misc_feature 1..6694: note = pAAV.CB7.CI.201IA.rBG
  regulatory 60..186: regulatory_class = polyA_signal_sequence
  misc_feature 275..404: note = 3' ITR (complement)
  rep_origin 581..1036
  misc_feature 3226..3355: note = 5' ITR
  misc_feature 3423..3804: note = misc - CMV IE promoter
  regulatory 3807..4088: regulatory_class = promoter
  regulatory 4061..4064: regulatory_class = TATA_box
  misc_feature 4183..5155: note = Intron
  source 1..6694: mol_type = other DNA
  CDS 5161..6690: note = 201IA
gtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggagttcgggctgagctgggtctttctggtggccctgctgaagggagtccagtgcgaggtgcagctgctggaatccggacctggcctggtgaaaccatctgagacactgagtctgacttgtgctgtctccggcctgtctatcagctccgatttctcctgggcatggattaggcagacccccggcaaggccctggaatatgtggggtacatccgcgggaacaccggagatacatactataatcctagtctgaagtcaaggctgactatctcaaaggacaccagcaaaaaccaaatctacctgaatctgtctagtgtcaccgctggcgatgccgccgtgtactattgcgcaagggaccgggtgtgcgacgatgactacggatactattacaccgaggtgtgcttcggcctggattcttgggggcagggaatcgtggtcacagtgtcaagcggcggaggaggcagcggaggaggagggtccggaggcgggggatctgcagaactggtcatgacacagtccccactgagcctgtccgtcgctccaggacagactgcatctattagttgtcgatcctctcagtccctggactatgctaacggcaatacctacctgtcttggtttcaccagcgaccaggacagccacctcggagactgatctatcagatttccaacagagattctggagtgcccgacaggttctcaggcagcggagcaggaactgagtttaccctgcgaatcagtcggatggaatcagatgacgtggggatctactactgcggacaggggaccacattcccacggacatttggacagggcactaaggtggagatcaaaacctgtggaggaggaagcaagccaccaacctgccctccatgtacatctcccgaactgctgggcgggcctagcgtgttcctgtttccccctaagcctaaagatacactgatgattagtagaaccccagaggtcacatgcgtggtcgtggacgtgtcccaggaagatcctgacgtgaagttcaactggtacgtgaatggcgccgaggtgcaccatgctcagactaaaccacgcgaaacccagtataatagtacataccgagtcgtgtcagtcctgacagtgactcaccaggattggctgaacggcaaggagtatacctgcaaggtgtctaacaaggccctgcccgcccctatccagaaaacaattagcaaggacaaagggcagccacgggaaccccaggtgtacactctgccaccctcaagagaggaactgactaagaaccaggtcagcctgacctgtctggtgaaaggcttctaccccagcgatatcgtcgtggagtgggaaagttcaggccagcctgagaatacttacaagactacccctccagtgctggatagcgacgggtcctatttcctgtacagcaagctgacagtggacaaatcccgctggcagcagggaaacgtcttttcctgttctgtgatgcatgaggccctgcacaatcattacacccagaagagtctgtcactgagccccggcaaatgag

SEQ ID NO: 6 - protein, 510 aa, synthetic construct
  REGION 1..510: note = Synthetic Construct
MEFGLSWVFLVALLKGVQCEVQLLESGPGLVKPSETLSLTCAVSGLSISSDFSWAWIRQTPGKALEYVGYIRGNTGDTYYNPSLKSRLTISKDTSKNQIYLNLSSVTAGDAAVYYCARDRVCDDDYGYYYTEVCFGLDSWGQGIVVTVSSGGGGSGGGGSGGGGSAELVMTQSPLSLSVAPGQTASISCRSSQSLDYANGNTYLSWFHQRPGQPPRRLIYQISNRDSGVPDRFSGSGAGTEFTLRISRMESDDVGIYYCGQGTTFPRTFGQGTKVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 7 - DNA, 6694 bp, synthetic construct
  misc_feature 1..6694: note = Plasmid encoding 201IA(I253A)mutant
  misc_feature 1..130: note = misc - 5' ITR
  misc_feature 198..579: note = CMV IE promoter
  regulatory 582..863: note = promoter - CB promoter; regulatory_class = promoter
  regulatory 836..839: regulatory_class = TATA_box
  misc_feature 958..1930: note = Intron - chicken beta-actin intron
  regulatory 3529..3655: note = polyA_signal - rabbit globin polyA; regulatory_class = polyA_signal_sequence
  misc_feature 3744..3873: note = 3' ITR (complement)
  rep_origin 4050..4505
  misc_feature 4636..5493: note = AP(R) marker
  rep_origin 5667..6255
  source 1..6694: mol_type = other DNA
  CDS 1936..3465: note = CMV IE promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggagttcgggctgagctgggtctttctggtggccctgctgaagggagtccagtgcgaggtgcagctgctggaatccggacctggcctggtgaaaccatctgagacactgagtctgacttgtgctgtctccggcctgtctatcagctccgatttctcctgggcatggattaggcagacccccggcaaggccctggaatatgtggggtacatccgcgggaacaccggagatacatactataatcctagtctgaagtcaaggctgactatctcaaaggacaccagcaaaaaccaaatctacctgaatctgtctagtgtcaccgctggcgatgccgccgtgtactattgcgcaagggaccgggtgtgcgacgatgactacggatactattacaccgaggtgtgcttcggcctggattcttgggggcagggaatcgtggtcacagtgtcaagcggcggaggaggcagcggaggaggagggtccggaggcgggggatctgcagaactggtcatgacacagtccccactgagcctgtccgtcgctccaggacagactgcatctattagttgtcgatcctctcagtccctggactatgctaacggcaatacctacctgtcttggtttcaccagcgaccaggacagccacctcggagactgatctatcagatttccaacagagattctggagtgcccgacaggttctcaggcagcggagcaggaactgagtttaccctgcgaatcagtcggatggaatcagatgacgtggggatctactactgcggacaggggaccacattcccacggacatttggacagggcactaaggtggagatcaaaacctgtggaggaggaagcaagccaccaacctgccctccatgtacatctcccgaactgctgggcgggcctagcgtgttcctgtttccccctaagcctaaagatacactgatggccagtagaaccccagaggtcacatgcgtggtcgtggacgtgtcccaggaagatcctgacgtgaagttcaactggtacgtgaatggcgccgaggtgcaccatgctcagactaaaccacgcgaaacccagtataatagtacataccgagtcgtgtcagtcctgacagtgactcaccaggattggctgaacggcaaggagtatacctgcaaggtgtctaacaaggccctgcccgcccctatccagaaaacaattagcaaggacaaagggcagccacgggaaccccaggtgtacactctgccaccctcaagagaggaactgactaagaaccaggtcagcctgacctgtctggtgaaaggcttctaccccagcgatatcgtcgtggagtgggaaagttcaggccagcctgagaatacttacaagactacccctccagtgctggatagcgacgggtcctatttcctgtacagcaagctgacagtggacaaatcccgctggcagcagggaaacgtcttttcctgttctgtgatgcatgaggccctgcacaatcattacacccagaagagtctgtcactgagccccggcaaatgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 8 - protein, 510 aa, synthetic construct
  REGION 1..510: note = Synthetic Construct
MEFGLSWVFLVALLKGVQCEVQLLESGPGLVKPSETLSLTCAVSGLSISSDFSWAWIRQTPGKALEYVGYIRGNTGDTYYNPSLKSRLTISKDTSKNQIYLNLSSVTAGDAAVYYCARDRVCDDDYGYYYTEVCFGLDSWGQGIVVTVSSGGGGSGGGGSGGGGSAELVMTQSPLSLSVAPGQTASISCRSSQSLDYANGNTYLSWFHQRPGQPPRRLIYQISNRDSGVPDRFSGSGAGTEFTLRISRMESDDVGIYYCGQGTTFPRTFGQGTKVEIKTCGGGSKPPTCPPCTSPELLGGPSVFLFPPKPKDTLMASRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 9 - DNA, 6694 bp, synthetic construct
  misc_feature 1..6694: note = engineered plasmid containining 201IA(H435) mutant
  misc_feature 275..404: note = 3' ITR (complement)
  rep_origin 2198..2786
  misc_feature 3226..3355
  misc_feature 3423..3804: note = CMV IE promoter
  regulatory 3807..4088: note = promoter - CB promoter; regulatory_class = promoter
  regulatory 4061..4064: note = TATA_signal - rabbit globin polyA; regulatory_class = TATA_box
  misc_feature 4183..5155: note = Intron - chicken beta-actin intron
  source 1..6694: mol_type = other DNA
  CDS 5161..6690: note = CMV IE promoter
gtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggagttcgggctgagctgggtctttctggtggccctgctgaagggagtccagtgcgaggtgcagctgctggaatccggacctggcctggtgaaaccatctgagacactgagtctgacttgtgctgtctccggcctgtctatcagctccgatttctcctgggcatggattaggcagacccccggcaaggccctggaatatgtggggtacatccgcgggaacaccggagatacatactataatcctagtctgaagtcaaggctgactatctcaaaggacaccagcaaaaaccaaatctacctgaatctgtctagtgtcaccgctggcgatgccgccgtgtactattgcgcaagggaccgggtgtgcgacgatgactacggatactattacaccgaggtgtgcttcggcctggattcttgggggcagggaatcgtggtcacagtgtcaagcggcggaggaggcagcggaggaggagggtccggaggcgggggatctgcagaactggtcatgacacagtccccactgagcctgtccgtcgctccaggacagactgcatctattagttgtcgatcctctcagtccctggactatgctaacggcaatacctacctgtcttggtttcaccagcgaccaggacagccacctcggagactgatctatcagatttccaacagagattctggagtgcccgacaggttctcaggcagcggagcaggaactgagtttaccctgcgaatcagtcggatggaatcagatgacgtggggatctactactgcggacaggggaccacattcccacggacatttggacagggcactaaggtggagatcaaaacctgtggaggaggaagcaagccaccaacctgccctccatgtacatctcccgaactgctgggcgggcctagcgtgttcctgtttccccctaagcctaaagatacactgatgattagtagaaccccagaggtcacatgcgtggtcgtggacgtgtcccaggaagatcctgacgtgaagttcaactggtacgtgaatggcgccgaggtgcaccatgctcagactaaaccacgcgaaacccagtataatagtacataccgagtcgtgtcagtcctgacagtgactcaccaggattggctgaacggcaaggagtatacctgcaaggtgtctaacaaggccctgcccgcccctatccagaaaacaattagcaaggacaaagggcagccacgggaaccccaggtgtacactctgccaccctcaagagaggaactgactaagaaccaggtcagcctgacctgtctggtgaaaggcttctaccccagcgatatcgtcgtggagtgggaaagttcaggccagcctgagaatacttacaagactacccctccagtgctggatagcgacgggtcctatttcctgtacagcaagctgacagtggacaaatcccgctggcagcagggaaacgtcttttcctgttctgtgatgcatgaggccctgcacaatgcttacacccagaagagtctgtcactgagccccggcaaatgag

SEQ ID NO: 10 - protein, 510 aa, synthetic construct
  REGION 1..510: note = Synthetic Construct
MEFGLSWVFLVALLKGVQCEVQLLESGPGLVKPSETLSLTCAVSGLSISSDFSWAWIRQTPGKALEYVGYIRGNTGDTYYNPSLKSRLTISKDTSKNQIYLNLSSVTAGDAAVYYCARDRVCDDDYGYYYTEVCFGLDSWGQGIVVTVSSGGGGSGGGGSGGGGSAELVMTQSPLSLSVAPGQTASISCRSSQSLDYANGNTYLSWFHQRPGQPPRRLIYQISNRDSGVPDRFSGSGAGTEFTLRISRMESDDVGIYYCGQGTTFPRTFGQGTKVEIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNAYTQKSLSLSPGK

SEQ ID NO: 11 - DNA, 7284 bp, synthetic construct
  misc_feature 1..7284: note = Plasmid containing the heavy and light chains of trastuzumab
  misc_feature 1..130: note = 5' ITR
  regulatory 191..932: note = promoter - human CMV IE enhancer and promoter; regulatory_class = promoter
  regulatory 897..901: regulatory_class = TATA_box
  misc_feature 1047..1179: note = Intron
  sig_peptide 1305..1364: note = IL-2 signal peptide
  regulatory 2726..3313: note = enhancer - IRES; regulatory_class = enhancer
  sig_peptide 3314..3373: note = IL2 signal peptide
  regulatory 4038..4269: note = polyA_signal - SV40 late polyA signal; regulatory_class = polyA_signal_sequence
  regulatory 4083..4269: note = polyA_signal - SV40 late polyA signal; regulatory_class = polyA_signal_sequence
  misc_feature 4334..4463: note = 3' ITR (complement)
  rep_origin 4640..5095
  misc_feature 6257..6845: note = COL/E1/origin
  source 1..7284: mol_type = other DNA
  CDS 1365..1724: note = Trastuzumab heavy variable
  CDS 1725..2720: note = trastuzumab heavy constant
  CDS 3374..3694: note = trastuzumab light variable
  CDS 3695..4021: note = trastuzumab constant light (kappa)
  CDS 5226..6083: note = amp-R
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggggactttgcactggaacttacaacacccgagcaaggacgcgactctagacccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaaggggccctcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggcaagtgataaggccggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgtaccgcatgcaactcctgtcttgcattgcactaagtcttgcacttgtcacaaacagtgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctgatgaaagcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 12 - protein, 120 aa, synthetic construct [sequence omitted]

SEQ ID NO: 13 - protein, 330 aa, synthetic construct [sequence omitted]

SEQ ID NO: 14 - protein, 107 aa, synthetic construct [sequence omitted]

SEQ ID NO: 15 - protein, 107 aa, synthetic construct [sequence omitted]

SEQ ID NO: 16 - protein, 286 aa, synthetic construct
  REGION 1..286: note = Synthetic Construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 17 - DNA, 4440 bp, synthetic construct
  misc_feature 1..4440: note = Engineered plasmid containing trastuzumab MAb
  sig_peptide 1254..1313: note = IL2 signal sequence
  sig_peptide 2745..2804: note = IL2 signal sequence
  source 1..4440: mol_type = other DNA
  CDS 1314..1673: note = Trastuzamab heavy variable
  CDS 1674..2660: note = Trastuzamab heavy constant
  CDS 2805..3110: note = Trastuzamab light variable
  CDS 3111..3452: note = Trastuzamab light constant (kappa)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagacccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggcagaaagcggagagcccccgtgaagcagaccctgaacttcgacctgctgaagctggccggcgacgtggaaagcaaccctggccctatgtacagaatgcagctgctgctgctgatcgccctgagcctggccctggtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccga

SEQ ID NO: 18 - protein, 120 aa, synthetic construct [sequence omitted]

SEQ ID NO: 19 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 102 aa, synthetic construct
  REGION 1..102: note = Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGT

SEQ ID NO: 21 - protein, 112 aa, synthetic construct
  REGION 1..112: note = Synthetic Construct
KVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 12 aa, synthetic construct
  REGION 1..12: note = Peptide mimotope of HER2 epitope
LLGPYELWELSH

SEQ ID NO: 23 - protein, 689 aa, synthetic construct
  REGION 1..689: note = trastuzumab immunoglobulin H435A mutant
GAGCCCAAATCTTGTGACAAAACTCACACATGCCCACCGTGCCCAGCACCTGAACTCCTGGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAGCCCCCATCGAGAAAACCATCTCCAAAGCCAAAGGGCAGCCCCGAGAACCACAGGTGTACACCCTGCCCCCATCCCGGGATGAGCTGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCTCCGTGATGCATGAGGCTCTGCACAACGCCTACACGCAGAAGAGCCTCTCCCTGTCTCC

SEQ ID NO: 24 - DNA, 689 bp, synthetic construct
  misc_feature 1..689: note = immunoglobulin fragment with I253A mutant
  source 1..689: mol_type = other DNA
gagcccaaatcttgtgacaaaactcacacatgcccaccgtgcccagcacctgaactcctggggggaccgtcagtcttcctcttccccccaaaacccaaggacaccctcatggcctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcc

SEQ ID NO: 25 - protein, 469 aa, synthetic construct
  REGION 1..469: note = Trastruzumab heavy chain
MYRMQLLSCIALSLALVTNSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2024/0277871 A1
(43) Pub. Date: Aug. 22, 2024

(54) METHODS AND COMPOSITIONS FOR TREATING METASTATIC BREAST CANCER AND OTHER CANCERS IN THE BRAIN

(71) Applicant: The Trustees of the University of Pennsylvania, Philadelphia, PA (US)

(72) Inventors: James M. Wilson, Philadelphia, PA (US); William Thomas Rothwell, Philadelphia, PA (US); Christian Hinderer, Baltimore, MD (US)

(73) Assignee: The Trustees of the University of Pennsylvania, Philadelphia, PA (US)

(21) Appl. No.: 18/519,972

(22) Filed: Nov. 27, 2023

Related U.S. Application Data
(63) Continuation of application No. 16/986,946, filed on Aug. 6, 2020, now Pat. No. 11,865,187.
Application No. 16/986,946 is a continuation of application No. 15/306,413, filed on Oct. 24, 2016, now Pat. No. 10,780,182.
(60) Provisional application No. 61/984,686, filed on Apr. 25, 2014.

(51) Int. Cl. A61K 48/00; A61K 9/00; C07K 16/32

(52) U.S. Cl. CPC A61K 48/005; A61K 9/0085; C07K 16/32; C07K 2317/24; C12N 2750/14143

(57) ABSTRACT

A composition comprising at least one AAV vector formulated for central nervous system delivery is described. The composition comprises at least one expression cassette which contains sequences encoding an anti-neoplastic immunoglobulin construct for delivery to the brain operably linked to expression control sequences therefor and a pharmaceutically acceptable carrier. The anti-neoplastic immunoglobulin construct may be an immunoglobulin modified to have decreased or no measurable affinity for neonatal Fc receptor (FcRn). Also provided are methods of using these constructs in preparing pharmaceutical compositions and uses thereof in anti-neoplastic regimens, particularly for primary and/or metastatic cancers of the brain.

### CROSS-REFERENCE TO RELATED APPLICATIONS

This is a continuation of U.S. patent application Ser. No. 16/986,946, filed Aug. 6, 2020, which is a continuation of U.S. patent application Ser. No. 15/306,413, filed Oct. 24, 2016, now U.S. Pat. No. 10,780,182, which is a National Stage Entry under 35 U.S.C. 371 of International Patent Application No. PCT/US2015/027491, filed Apr. 24, 2015, now expired, and which claims benefit under 35 USC 119(e) of U.S. Provisional Patent Application No. 61/984,686, filed Apr. 25, 2014, now expired. These applications are incorporated by reference in their entirety.

### REFERENCE TO AN ELECTRONIC SEQUENCE LISTING

The electronic sequence listing filed herewith named “UPN-14-7028USC2.xml” with size of 98,298 bytes, created on date of Nov. 27, 2023, and the contents of the electronic sequence listing (e.g., the sequences and text therein) are incorporated herein by reference in entirety.

### BACKGROUND OF THE INVENTION

Brain metastases are a common and devastating sequelae of breast cancer for which treatment options are few and inadequate. 6-16% of breast cancer patients develop central nervous system (CNS) metastases. These patients have a 20% one-year and 1.3% five-year median survival from the time of diagnosis. DiStefano A, et al, Cancer. 1979; 44:1913-1918; Takakura K, et al, Metastatic tumors of the central nervous system. Tokyo: Igaku-Shoin, 1982; Hall W A, et al, Long-term survival with metastatic cancer to the brain. Med Oncol. 2000 November; 17(4):279-86; Pieńkowski T, Zielinski C C. Trastuzumab treatment in patients with breast cancer and metastatic CNS disease. Ann Oncol. 2010 May; 21(5):917-24. Surgical resection of brain metastases is often infeasible, and chemotherapeutic agents are mostly excluded from the CNS by the blood brain barrier (BBB) [Nakayama A, et al, Antitumor Activity of TAK-285, an Investigational, Non-Pgp Substrate HER2/EGFR Kinase Inhibitor, in Cultured Tumor Cells, Mouse and Rat Xenograft Tumors, and in an HER2-Positive Brain Metastasis Model, J Cancer. 2013 Aug. 16; 4(7)]. Alternative therapies to treat breast cancer brain metastases are needed.

Breast cancers that overexpress the HER2 receptor tyrosine kinase have a high propensity to metastasize to the CNS and comprise 25-30% of all breast cancer cases [Bendell J C, et al, Central nervous system metastases in women who receive trastuzumab-based therapy for metastatic breast carcinoma. Cancer. 2003 Jun. 15; 97(12):2972-7]. Trastuzumab (Herceptin®) is a first-line therapeutic immunoglobulin G (IgG) monoclonal antibody (mAb) directed toward HER2; this antibody has been reported to significantly improve survival of patients with HER2 positive disease [Lin N U, et al, Brain metastases: the HER2 paradigm. Clin Cancer Res. 2007 Mar. 15; 13(6):1648-55; Palmieri D, et al, Her-2 overexpression increases the metastatic outgrowth of breast cancer cells in the brain. Cancer Res. 2007 May 1; 67(9):4190-8]. However, patients that benefit from trastuzumab often experience simultaneous progression of CNS disease because mAbs do not cross the BBB [Nakayama, cited above]. Injecting trastuzumab directly into the CNS has been proven to be safe, and intrathecal administration of trastuzumab to patients with leptomeningeal carcinomatosis has been reported to increase overall survival from 2 to 13.5 months [Zagouri F, et al, Intrathecal administration of trastuzumab for the treatment of meningeal carcinomatosis in HER2-positive metastatic breast cancer: a systematic review and pooled analysis. Breast Cancer Res Treat. 2013 May; 139(1):13-22]. Leptomeningeal carcinomatosis is associated with an impaired, rather than an intact, blood-brain barrier. Park, E-J, et al, J Controlled Release, 163 (2012), 277-284 report that focused ultrasound bursts combined with circulating microbubbles can temporarily permeabilize both the blood brain barrier and the blood tumor barrier for trastuzumab.

While current therapies have led to an improved control of the systemic disease, treatment of metastatic dissemination of human breast cancer into the CNS is a great therapeutic challenge.

### SUMMARY OF THE INVENTION

An anti-neoplastic composition is provided which comprises at least one AAV vector formulated for delivery to the central nervous system, wherein said composition comprises at least one expression cassette which contains sequences encoding an anti-neoplastic immunoglobulin product for delivery to the CNS operably linked to expression control sequences therefor and a pharmaceutically acceptable carrier. In one example, the anti-neoplastic immunoglobulin construct comprises an immunoglobulin modified to have decreased or no measurable affinity for neonatal Fc receptor (FcRn). Suitably, this composition is effective for use in retarding the growth of a tumor in the brain and/or for reducing tumor size and/or for increasing progression-free survival of the subject.

In one aspect, a composition as provided herein comprises an AAV viral vector having an AAV9 capsid and having packaged therein an expression cassette encoding an anti-Her2 IgG antibody or a functional fragment thereof which comprises an anti-Her2 heavy chain which has disrupted binding for FcRn.

In another aspect, a method for retarding the growth of a tumor in the brain is provided, which involves administering a composition as described herein to the central nervous system e.g., intrathecally. In one aspect, the composition is administered in the absence of chemical or physical disruption of the blood brain barrier.

In yet another aspect, the invention provides a method for treating a neoplasm in the brain by administering a composition as described herein to a subject in need thereof.

In yet another aspect, the invention provides an anti-neoplastic regimen comprising administering a composition as described herein in combination with an antibody or other biologic drug, a small molecule anti-neoplastic agent, radiation, and/or a chemotherapeutic agent.

Still other aspects and advantages of the invention will be readily apparent from the following detailed description of the invention.

### BRIEF DESCRIPTION OF THE DRAWINGS

FIG. 1 provides the amino acid sequence of the heavy chain of the polypeptide of trastuzumab, with the sequence listing numbering [SEQ ID NO:25] above the sequence and the conventional Eu (IMGT) numbering below the sequence.

FIG. 2 provides a survival curve of mice given 1×10¹¹ GC ICV of AAV9.trastuzumab or AAV9.201IA prophylactically, then implanted with BT474-M1.ffluc tumor cells in the brain 21 days after vector administration. The median survival of 201IA group (sham treatment) is shown to be 66 days, whereas the median survival of the group treated with AAV9.trastuzumab is 99 days, a 33% increase in survival rate.

### DETAILED DESCRIPTION OF THE INVENTION

The compositions and regimens described herein are useful for delivery of anti-neoplastic immunoglobulin constructs to the central nervous system. Compositions described herein comprising AAV-Ig are well suited for central nervous system (CNS) cancers (neoplasms), and particularly for those located in the brain.

As used herein, the term “CNS neoplasms” includes primary or metastatic cancers, which may be located in the brain (intracranial), meninges (connective tissue layer covering brain and spinal cord), or spinal cord. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

As used herein, an “anti-neoplastic” immunoglobulin construct (including antibody or antibody fragment as defined herein) encodes a polypeptide-based moiety which binds to a cell-surface antigen or receptor located on a cancer cell or solid tumor and which inhibits or prevents the growth and spread of tumors, or malignant cells in a non-solid tumor, and optionally, reduces the size of tumors. The anti-neoplastic immunoglobulin polypeptides can function by a number of mechanisms, e.g., inhibiting tumor cell growth by blocking a growth factor receptor, cross-linking cell membrane antigens to deliver signals that control the cell cycle, blocking angiogenesis, blocking DNA repair post chemotherapy, or even inducing cell death. Alternatively, they can influence tumor growth indirectly by activating host immune effector functions such as antibody-dependent and complement-mediated cell cytotoxicity. In one embodiment, the anti-neoplastic effect of the compositions and regimens described herein can be measured by reduction of tumor size and/or by an increased progression-free survival rate as compared to subjects which are untreated or treated with other regimens.

The term “immunoglobulin” is used herein to include antibodies, functional fragments thereof, and immunoadhesins. Antibodies may exist in a variety of forms including, for example, polyclonal antibodies, monoclonal antibodies, camelized single domain antibodies, intracellular antibodies (“intrabodies”), recombinant antibodies, multispecific antibody, antibody fragments, such as, Fv, Fab, F(ab)₂, F(ab)₃, Fab′, Fab′-SH, F(ab′)₂, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), chimeric antibodies, chimeric antibodies comprising at least one human constant region, and the like. “Antibody fragment” refers to at least a portion of the variable region of the immunoglobulin that binds to its target, e.g., the tumor cell.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

As used herein, an “expression cassette” refers to a nucleic acid molecule which comprises an immunoglobulin gene(s) (e.g., an immunoglobulin variable region, an immunoglobulin constant region, a full-length light chain, a full-length heavy chain or another fragment of an immunoglobulin construct), promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the immunoglobulin sequences described herein flanked by packaging signals of the viral genome and other expression control sequences such as those described herein.

As described above, the term “about” when used to modify a numerical value means a variation of ±10%, unless otherwise specified.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

For expression from an AAV vector, the amino acid sequences for an anti-neoplastic immunoglobulin construct are selected from those which have been published, those which are commercially available, and the coding sequences described herein. Anti-neoplastic immunoglobulins as described herein may target a human epidermal growth factor receptor (HER), such as HER2. An example of trastuzumab is a recombinant IgG1 kappa, humanized monoclonal antibody that selectively binds with high affinity in a cell-based assay (Kd=5 nM) to the extracellular domain of the human epidermal growth factor receptor protein. The commercially available product is produced in CHO cell culture. See, e.g., web site: drugbank.ca/drugs/DB00072. The amino acid sequences of the trastuzumab light chains 1 and 2 and heavy chains 1 and 2, as well as sequences obtained from a study of the x-ray structure of trastuzumab, are provided on this database at accession number DB00072, which sequences are incorporated herein by reference. See, also, 212-Pb-TCMC-trastuzumab [Areva Med, Bethesda, MD]. Another antibody of interest includes, e.g., pertuzumab, a recombinant humanized monoclonal antibody that targets the extracellular dimerization domain (Subdomain II) of the human epidermal growth factor receptor 2 protein (HER2). It consists of two heavy chains and two lights chains that have 448 and 214 residues respectively. FDA approved Jun. 8, 2012. The amino acid sequences of its heavy chain and light chain are provided, e.g., in web site: drugbank.ca/drugs/DB06366 (synonyms include 2C4, MOAB 2C4, monoclonal antibody 2C4, and rhuMAb-2C4) on this database at accession number DB06366. In addition to HER2, other HER targets may be selected.

For example, MM-121/SAR256212 is a fully human monoclonal antibody that targets the HER3 receptor [Merrimack's Network Biology] and which has been reported to be useful in the treatment of non-small cell lung cancer (NSCLC), breast cancer and ovarian cancer. SAR256212 is an investigational fully human monoclonal antibody that targets the HER3 (ErbB3) receptor [Sanofi Oncology]. Another anti-Her3/EGFR antibody is RG7597 [Genentech], described as being useful in head and neck cancers. Another antibody, margetuximab (or MGAH22), a next-generation, Fc-optimized monoclonal antibody (mAb) that targets HER [MacroGenics], may also be utilized.

Alternatively, other human epithelial cell surface markers and/or other tumor receptors or antigens may be targeted. Examples of other cell surface marker targets include, e.g., 5T4, CA-125, CEA (e.g., targeted by labetuzumab), CD3, CD19, CD20 (e.g., targeted by rituximab), CD22 (e.g., targeted by epratuzumab or veltuzumab), CD30, CD33, CD40, CD44, CD51 (also integrin αᵥβ₃), CD133 (e.g., glioblastoma cells), CTLA-4 (e.g., Ipilimumab used in treatment of, e.g., neuroblastoma), Chemokine (C—X—C Motif) Receptor 2 (CXCR2) (expressed in different regions in brain; e.g., Anti-CXCR2 (extracellular) antibody #ACR-012 (Alomene Labs)); EpCAM, fibroblast activation protein (FAP) [see, e.g., WO 2012020006 A2, brain cancers], folate receptor alpha (e.g., pediatric ependymal brain tumors, head and neck cancers), fibroblast growth factor receptor 1 (FGFR1) (see, et al, WO2012125124A1 for discussion treatment of cancers with anti-FGFR1 antibodies), FGFR2 (see, e.g., antibodies described in WO2013076186A and WO2011143318A2), FGFR3 (see, e.g., antibodies described in U.S. Pat. No. 8,187,601 and WO2010111367A1), FGFR4 (see, e.g., anti-FGFR4 antibodies described in WO2012138975A1), hepatocyte growth factor (HGF) (see, e.g., antibodies in WO2010119991A3), integrin α₅β₁, IGF-1 receptor, gangioloside GD2 (see, e.g., antibodies described in WO2011160119A2), ganglioside GD3, transmembrane glycoprotein NMB (GPNMB) (associated with gliomas, among others and target of the antibody glembatumumab (CR011), mucin, MUC1, phosphatidylserine (e.g., targeted by bavituximab, Peregrine Pharmaceuticals, Inc], prostatic carcinoma cells, PD-L1 (e.g., nivolumab (BMS-936558, MDX-1106, ONO-4538), a fully human gG4, e.g., metastatic melanoma], platelet-derived growth factor receptor, alpha (PDGFR α) or CD140, tumor associated glycoprotein 72 (TAG-72), tenascin C, tumor necrosis factor (TNF) receptor (TRAIL-R2), vascular endothelial growth factor (VEGF)-A (e.g., targeted by bevacizumab) and VEGFR2 (e.g., targeted by ramucirumab). Other antibodies and their targets include, e.g., APN301 (hul4.19-IL2), a monoclonal antibody [malignant melanoma and neuroblastoma in children, Apeiron Biologics, Vienna, Austria]. See, also, e.g., monoclonal antibody, 8H9, which has been described as being useful for the treatment of solid tumors, including metastatic brain cancer. The monoclonal antibody 8H9 is a mouse IgG1 antibody with specificity for the B7H3 antigen [United Therapeutics Corporation]. This mouse antibody can be humanized. Still other immunoglobulin constructs targeting the B7-H3 and/or the B7-H4 antigen may be used in the invention. Another antibody is S58 (anti-GD2, neuroblastoma). Cotara™ [Perregrince Pharmaceuticals] is a monoclonal antibody described for treatment of recurrent glioblastoma. Other antibodies may include, e.g., avastin, ficlatuzumab, medi-575, and olaratumab. Still other immunoglobulin constructs or monoclonal antibodies may be selected for use in the invention. See, e.g., Medicines in Development Biologics, 2013 Report, pp. 1-87, a publication of PhRMA's Communications & Public Affairs Department. (202) 835-3460, which is incorporated by reference herein.

Once the target and immunoglobulin are selected, the coding sequences for the selected immunoglobulin (e.g., heavy and/or light chain(s)) may be obtained and/or synthesized. Methods for sequencing a protein, peptide, or polypeptide (e.g., as an immunoglobulin) are known to those of skill in the art. Once the sequence of a protein is known, there are web-based and commercially available computer programs, as well as service based companies which back translate the amino acids sequences to nucleic acid coding sequences. See, e.g., backtranseq by EMBOSS, world wide web at: ebi_ac_.uk/Tools/st/; Gene Infinity world wide web @geneinfinity_org/sms/sms_backtranslation.html); ExPasy (world wide web: expasy_org/tools/). In one embodiment, the RNA and/or cDNA coding sequences are designed for optimal expression in human cells.

Codon-optimized coding regions can be designed by various different methods. This optimization may be performed using methods which are available on-line (e.g., GeneArt), published methods, or a company which provides codon optimizing services, e.g., DNA2.0 (Menlo Park, CA). One codon optimizing algorithm is described, e.g., in US International Patent Publication No. WO 2015/012924, which is incorporated by reference herein. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

The immunoglobulin genes described herein may be used to express the “wild-type”, a published or commercially available, or other known constant immunoglobulin domains or can be engineered to decrease affinity for, or ablate, binding to the Fc binding site present on immunoglobulins. There are several different types of Fc receptors, which are classified based on the type of antibody that they recognize. As used herein, “FcRn” refers to the neonatal Fc receptor that binds IgG. It is similar in structure to MHC class I protein. In humans, it is encoded by the FCGRT gene. The Fc receptor is located on various cells types, including, e.g., the epithelial cells of the blood brain barrier. The term “FcRn binding domain” as used herein refers to a protein domain that directly or indirectly binds to the FcRn. The FcRn may be a mammalian FcRn. In further embodiments, the FcRn is a human FcRn. An FcRn binding domain binding directly to an FcRn is an antibody Fc region. Meanwhile, regions capable of binding to a polypeptide such as albumin or IgG, which has human FcRn-binding activity, can indirectly bind to human FcRn via albumin, IgG, or such. Thus, such a human FcRn-binding region may be a region that binds to a polypeptide having human FcRn-binding activity. The term “Fc region” as used herein refers to an FcRn-binding domain that directly binds to FcRn, a mammalian FcRn, or a human FcRn. In particular, an Fc region is an Fc region of an antibody. The Fc region may be a mammalian Fc region or more particularly a human Fe region. In particular, the Fe region may be located within the second and third constant domain of a human immunoglobulin (CH2 and CH3). Further, the Fc region may be the hinge of CH2 and CH3. In one embodiment, the immunoglobulin construct is an IgG. In a further embodiment, the Fc region is an Fc region of human IgG1. Other Ig isotypes can be used as well.

Because these binding domains are located within the constant region of an IgG heavy chain (regions CH2 and CH3), the amino acid positions provided herein for modification in trastuzumab can be readily determined by preparing an alignment with another immunoglobulin heavy chain selected for modification in order to identify the corresponding amino acid number. Methods and computer programs for preparing such alignments are available and well known to those of skill in the art. The amino acid positions referred to in this application are based upon the numbering of trastuzumab as provided in SEQ ID NO: 3 and 25 (heavy chain) and SEQ ID NO: 4 (light chain). Substitutions may also be written as (amino acid identified by single letter code)—position #—(amino acid identified by single letter code) whereby the first amino acid is the substituted amino acid and the second amino acid is the substituting amino acid at the specified position. The terms “substitution” and “substitution of an amino acid” and “amino acid substitution” as used herein refer to a replacement of an amino acid in an amino acid sequence with another one, wherein the latter is different from the replaced amino acid. Methods for replacing an amino acid are well known to the skilled in the art and include, but are not limited to, mutations of the nucleotide sequence encoding the amino acid sequence. Methods of making amino acid substitutions in IgG are described, e.g., for WO 2013/046704, which is incorporated by reference for its discussion of amino acid modification techniques, although this document describes increasing FcRn affinity, rather than decreasing or ablating binding affinity as described herein.

The term “amino acid substitution” and its synonyms described above are intended to encompass modification of an amino acid sequence by replacement of an amino acid with another, substituting amino acid. The substitution may be a conservative substitution. The term conservative, in referring to two amino acids, is intended to mean that the amino acids share a common property recognized by one of skill in the art. The term non-conservative, in referring to two amino acids, is intended to mean that the amino acids which have differences in at least one property recognized by one of skill in the art. For example, such properties may include amino acids having hydrophobic nonacidic side chains, amino acids having hydrophobic side chains (which may be further differentiated as acidic or nonacidic), amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Thus, a conservative amino acid substitution may involve changing a first amino acid having a hydrophobic side chain with a different amino acid having a hydrophobic side chain; whereas a non-conservative amino acid substitution may involve changing a first amino acid with an acidic hydrophobic side chain with a different amino acid having a different side chain, e.g., a basic hydrophobic side chain or a hydrophilic side chain. Still other conservative or non-conservative changes change be determined by one of skill in the art.

In still other embodiments, the substitution at a given position will be to an amino acid, or one of a group of amino acids, that will be apparent to one of skill in the art in order to accomplish an objective identified herein.

In one embodiment, an immunoglobulin construct as defined herein is engineered so that the native sequence located on the conserved region of the immunoglobulin Fc region is ablated to eliminate binding to the FcRn and to minimize or eliminate transport of the proteinaceous immunoglobulin constructs across the blood brain barrier (out of the CNS area) and into the systemic circulation. In one example, this may be accomplished by altering one or more amino acids of the FcRn-binding domain, e.g., by modification of the codon for the selected amino acid(s).

For example, the immunoglobulin may be modified in one or more of the codons encoding the amino acid reside at position Y436 (aa459 of SEQ ID NO: 25), S254 (aa277 of SEQ ID NO:25), I253 (aa276 of SEQ ID NO: 25), and/or H435 (aa458 of SEQ ID NO: 25) to another suitable amino acid, e.g., alanine (Ala, A). However, other positions involved in functionally binding to FcRn may be mutated, such as, e.g., T250 (aa 273 of SEQ ID NO:25), M252 (aa 275 of SEQ ID NO: 25), S254 (aa 277 of SEQ ID NO: 25), T256 (aa 279 of SEQ ID NO: 25), P257 (aa 280 of SEQ ID NO: 25), P271 (aa 294 of SEQ ID NO:25), T307 (aa 330 of SEQ ID NO:25), Q311 (aa 334 of SEQ ID NO: 25), D376 (aa 399 of SEQ ID NO: 25), E380 (aa 403 of SEQ ID NO: 25), M428 (aa 451 of SEQ ID NO: 25), and/or N434 (aa 457 of SEQ ID NO: 25), or combinations one or more of these with each other, or with the other modifications described herein. Other suitable modifications may be located at 1253 (aa 276 of SEQ ID NO: 25), S254 (aa 277 of SEQ ID NO: 25), K288 (aa 311 of SEQ ID NO: 25), V305 (aa 328 of SEQ ID NO: 25), Q311 (aa 334 of SEQ ID NO: 25), D312 (aa 335 of SEQ ID NO: 25), K317 (aa 340 of SEQ ID NO: 340), K360 (aa 383 of SEQ ID NO: 25), Q362 (aa 385 of SEQ ID NO: 25), E380 (aa 403 of SEQ ID NO: 25), S415 (aa 438 of SEQ ID NO: 25), S424 (aa 447 of SEQ ID NO: 25), H433 (aa 456 of SEQ ID NO: 25), N434 (aa 457 of SEQ ID NO: 25), H435 (aa 458 of SEQ ID NO: 25), and/or Y436 (aa 459 of SEQ ID NO: 25), or combinations of two or more. As described above, corresponding locations in other IgG heavy chain CH2 and CH2 may be selected. Reference to “one or more” herein is intended to encompass the individual embodiments of, for example, 1, 2, 3, 4, 5. In additional embodiments, the term “one or more” includes a number of substitutions in a polypeptide described herein that would yield at least about 85% identity, at least 90% identity, at least about 95% identity, or at least about 99% identity to the trastuzumab heavy chain variable region SEQ ID NO: 3, light chain variable region SEQ ID NO:4, heavy chain SEQ ID NO: 25 or another amino acid sequence identified herein.

In addition, mutations that enhance complement-dependent cytotoxicity (CDC) and/or antibody-dependent cell-mediated cytotoxicity (ADCC) functions may be incorporated in the trastuzumab variants described herein. In further embodiments, such a mutation facilitates the killing of the tumor cells by immune cells. Examples of suitable amino acid modifications to enhance ADCC function are described in, e.g., US Patent Publication No. 2008/0118501; A Nasume, et al, Drug Des Devel Ther, 2009, 3; 7-16, publ online Sep. 21, 2009. GA Lazar et al, Proc Natl Acad Sci, vol. 103, no. 11, p. 4005-4010 (Mar. 14, 2006); and GL Moore, et al, MAbs, 2010 March-April; 2(2): 181-189, among others.

The heavy chain amino acid numbering used herein to identify the location of the mutants is based on the EU numbering system [IMGT unique numbering, Edelman, G. M. et al., Proc. Natl. Acad. USA, 63, 78-85 (1969); web site: imgt_.org/IMGTScientificChart/-Numbering/Hu_IGHGnber_html] ] and refer to positions in an FcRn-binding domain, in particular in an Fc region. In a similar fashion, substitutions are indicated as for example “EU387R” or “EU440E”, wherein the number given after “EU” indicates the position of the substitution according to the EU numbering, and the letter after the number is the substituted amino acid given in the one letter code. Other numbering systems include, e.g., Kabat, E. A., T. T. Wu, H. M. Perry, K. S. Gottesman, C. Foeler. (1991) Sequences of Proteins of Immunological Interest. No. 91-3242 U. S. Public Health Services, National Institutes of Health, Bethesda).

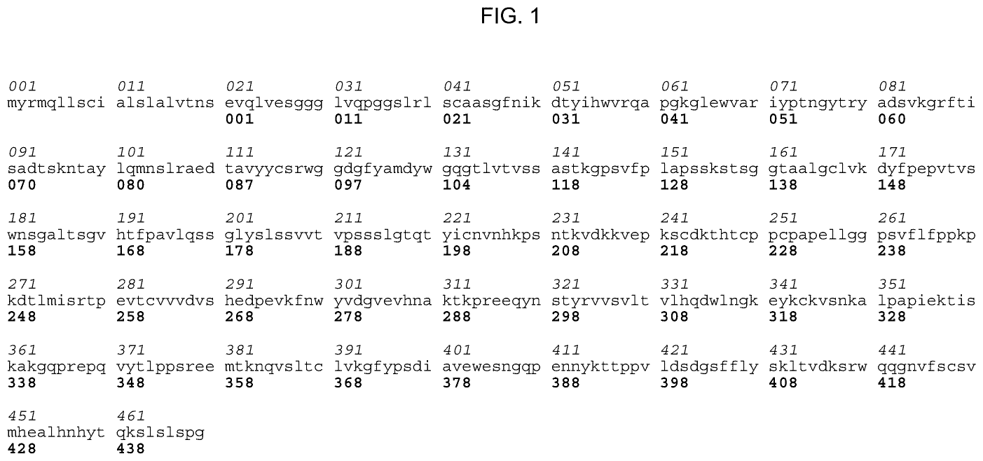

In one embodiment, an anti-Her2 antibody is selected for a composition and method as described herein. In one embodiment, the selected antibody is trastuzumab. The amino acid sequences of trastuzumab have been described, e.g., in P. Carter et al, Proc Natl Acad Sci., 89:4285-4289 (May 1982). The amino acid sequence of the trastuzumab heavy chain is provided in FIG. 1, showing both the sequence listing [SEQ ID NO: 25] and EU numbering systems. The amino acid sequence of the trastuzumab heavy chain variable region is shown in [SEQ ID NO: 3] and the trastuzumab light chain variable region is provided in the appended sequence listing [SEQ ID NO: 4]. In order to express trastuzumab, a novel nucleic acid molecule has been designed which contains codons which have been selected for optimal expression of the trastuzumab polypeptides in humans. Further, the novel nucleic acid molecule includes a heterologous leader sequence for each the heavy chain and light chain of trastuzumab, which encodes the IL-2 signal leader peptide fused upstream of the heavy and chain polypeptides composed of the variable and constant regions. However, another heterologous leader sequence may be substituted for one or both of the IL-2 signal/leader peptide. Signal/leader peptides may be the same or different for each the heavy chain and light chain immunoglobulin constructs. These may be signal sequences which are natively found in an immunoglobulin (e.g., IgG), or may be from a heterologous source. Such heterologous sources may be a cytokine (e.g., IL-2, IL12, IL18, or the like), insulin, albumin, β-glucuronidase, alkaline protease or the fibronectin secretory signal peptides, amongst others. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

The expression cassette described herein may contain at least one internal ribosome binding site, i.e., an IRES, located between the coding regions of the heavy and light chains. Alternatively, the heavy and light chain may be separated by a furin-2a self-cleaving peptide linker [see, e.g., Radcliffe and Mitrophanous, Gene Therapy (2004), 11, 1673-1674. The expression cassette may contain at least one enhancer, i.e., CMV enhancer. Still other enhancer elements may include, e.g., an apolipoprotein enhancer, a zebrafish enhancer, a GFAP enhancer element, and brain specific enhancers such as described in WO 2013/1555222, woodchuck post hepatitis post-transcriptional regulatory element. Additionally, or alternatively, other, e.g., the hybrid human cytomegalovirus (HCMV)-immediate early (IE)-PDGR promoter or other promoter—enhancer elements may be selected. To enhance expression the other elements can be introns (like promega intron or chimeric chicken globin-human immunoglobulin intron).

As provided herein, with respect to the numbering of the engineered nucleic acid molecule in SEQ ID NO:1, a nucleic acid sequence encoding the heavy chain polypeptide of trastuzumab is characterized by the leader sequence (1-60 of SEQ ID NO: 1), nucleic acids 61 to 423 are the coding region for the immunoglobulin heavy chain (HC) variable sequence, nucleic acids 439 to 714 are the coding region for the HC constant region 1, nucleic acids 715 to 1410 are the coding region for the HC constant regions 2 and 3. The IRES is located at nucleic acids 1422-2012 of SEQ ID NO: 1 between the trastuzumab heavy chain and the leader sequence of the trastuzumab light chain coding sequence. The variable region of the trastuzumab light chain variable sequence is at nucleotides 2070-2391 of SEQ ID NO: 1; the light chain constant region is located at nucleic acids 2407 to 2711 of SEQ ID NO: 1.

Also encompassed herein are nucleic acid sequences encoding the trastuzumab immunoglobulin polypeptides [e.g., the heavy chain, the light chain, or fragments thereof, which fragments may include, e.g., complementarity determining regions (CDR) 1, 2 and/or 3, a constant region (1, 2, or 3) of SEQ ID NO: 1, or a sequence which is at least about 85% identical thereto, at least about 90%, at least about 95% identical thereto, or at least about 99% identical thereto to SEQ ID NO: 1, or a fragment thereof coding for an immunoglobulin polypeptide (e.g., the heavy chain, the light chain, or fragments thereof [e.g., the heavy chain, the light chain, or fragments thereof (e.g., a variable region (including, e.g., complementarity determining regions (CDR) 1, 2 and/or 3), a constant region (1, 2, or 3))], which encode polypeptides and fragments thereof having the same amino acid sequence as provided herein for the trastuzumab without any FcRN modifications.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., any one of the modified ORFs provided herein when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). As another example, polynucleotide sequences can be compared using Fasta, a program in GCG Version 6.1. Fasta provides alignments and percent sequence identity of the regions of the best overlap between the query and search sequences. For instance, percent sequence identity between nucleic acid sequences can be determined using Fasta with its default parameters (a word size of 6 and the NOPAM factor for the scoring matrix) as provided in GCG Version 6.1, herein incorporated by reference. Generally, these programs are used at default settings, although one skilled in the art can alter these settings as needed. Alternatively, one of skill in the art can utilize another algorithm or computer program that provides at least the level of identity or alignment as that provided by the referenced algorithms and programs. This definition also refers to, or can be applied to, the compliment of a sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences.

Typically, when an alignment is prepared based upon an amino acid sequence, the alignment contains insertions and deletions which are so identified with respect to a reference AAV sequence and the numbering of the amino acid residues is based upon a reference scale provided for the alignment. However, any given AAV sequence may have fewer amino acid residues than the reference scale. In the present invention, when discussing the parental sequence, the term “the same position” or the “corresponding position” refers to the amino acid located at the same residue number in each of the sequences, with respect to the reference scale for the aligned sequences. However, when taken out of the alignment, each of the proteins may have these amino acids located at different residue numbers. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

In another embodiment, a modified anti-Her2 antibody having its affinity for FcRn ablated and retaining effective anti-neoplastic activity is provided. One or more amino acid modifications may be selected to ablate functional binding to FcRn. In one embodiment, the mutation lowers the binding affinity of the trastuzumab immunoglobulin for FcRn to less than 10% of the native protein. Suitably, the immunoglobulins with these mutations bind substantially normally to all other Fc receptors. For example, the immunoglobulin may be modified at least one of position Y436, S254, 1253, and/or H435 to an alanine or other amino acid, or combinations of one or more of these with each other, or one, two or more with one or more of the modifications described herein. However, other positions involved in functional binding to FcRn may be mutated, such as, e.g., T250 (aa 273 of SEQ ID NO:25), M252 (aa 275 of SEQ ID NO: 25), S254 (aa 278 of SEQ ID NO: 25), T256 (aa 280 of SEQ ID NO: 25), P257 (aa 281 of SEQ ID NO: 25), P271 (aa 294 of SEQ ID NO:25), T307 (aa 330 of SEQ ID NO:25), Q311 (aa 334 of SEQ ID NO: 25), D376 (aa 399 of SEQ ID NO: 25), E380 (aa 403 of SEQ ID NO: 25), M428 (aa 451 of SEQ ID NO: 25), and/or N434 (aa 457 of SEQ ID NO: 25), or combinations one or more of these with each other, or with the other modifications described herein. Other suitable modifications may be located at 1253 (aa 276 of SEQ ID NO: 25), S254 (aa 278 of SEQ ID NO: 25), K288 (aa 311 of SEQ ID NO: 25), V305 (aa 328 of SEQ ID NO: 25), Q311 (aa 334 of SEQ ID NO: 25), D312 (aa 335 of SEQ ID NO: 25), K317 (aa 340 of SEQ ID NO: 340), K360 (aa 383 of SEQ ID NO: 25), Q362 (aa 385 of SEQ ID NO: 25), E380 (aa 403 of SEQ ID NO: 25), S415 (aa 438 of SEQ ID NO: 25), S424 (aa 447 of SEQ ID NO: 25), H433 (aa 456 of SEQ ID NO: 25), N434 (aa 457 of SEQ ID NO: 25), H435 (aa 458 of SEQ ID NO: 25), and/or Y436 (aa 459 of SEQ ID NO: 25), or combinations of two or more, e.g., by substitution with another amino acid which retains the desired anti-neoplastic activity. Still other mutations may be incorporated. See, e.g., Kuo and Aveson, mAbs, 3:5, 422-430 (September/October 2011) and Shield, J Biol Chem, 2001, 276: 659-6604. Once the amino acid sequence is selected, the nucleic acid sequences can be designed and/or the previously described sequences may be engineered as described above. These modifications are made by engineering the nucleic acid coding region using site directed mutagenesis or other genetic engineering techniques which are known to those of skill in the art.

Similar modifications may be engineered into another selected anti-HER2 immunoglobulin construct, or alternately, into another anti-neoplastic immunoglobulin construct as described herein.

In one embodiment, the immunoglobulin genes described herein are engineered into a genetic element (e.g., a plasmid) useful for generating AAV vectors which transfer the immunoglobulin construct sequences carried thereon. The selected vector may be delivered to a an AAV packaging cell by any suitable method, including transfection, electroporation, liposome delivery, membrane fusion techniques, high velocity DNA-coated pellets, viral infection and protoplast fusion. Stable packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

### AAV Vectors

An AAV vector as described herein can comprise one or more nucleic acid sequences, each of which encodes one or more of the heavy and/or light chain polypeptides, or other polypeptides, of an anti-neoplastic immunoglobulin construct. Suitably, a composition contains one or more AAV vectors which contain all of the polypeptides which form an anti-neoplastic construct in vivo. For example, a full-length antibody consists of four polypeptides: two identical copies of a heavy (H) chain polypeptide and two copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. In this respect, an AAV vector as described herein can comprise a single nucleic acid sequence that encodes the two heavy chain polypeptides (e.g., constant variable) and the two light chain polypeptides of an immunoglobulin construct. Alternatively, the AAV vector can comprise a first expression cassette that encodes at least one heavy chain constant polypeptides and at least one heavy chain variable polypeptide, and a second expression cassettes that encodes both light chain polypeptides of an immunoglobulin construct. In yet another embodiment, the AAV vector can comprise a first expression cassette encoding a first heavy chain polypeptide, a second expression cassette encoding a second heavy chain polypeptide, a third expression cassette encoding a first light chain polypeptide, and a fourth expression cassette encoding a second light chain polypeptide.

Typically, an expression cassette for an AAV vector comprises an AAV5′ inverted terminal repeat (ITR), the immunoglobulin construct coding sequences and any regulatory sequences, and an AAV3′ ITR. However, other configurations of these elements may be suitable. A shortened version of the 5′ ITR, termed ΔITR, has been described in which the D-sequence and terminal resolution site (trs) are deleted. In other embodiments, the full-length AAV5′ and 3′ ITRs are used.

Where a pseudotyped AAV is to be produced, the ITRs in the expression are selected from a source which differs from the AAV source of the capsid. For example, AAV2 ITRs may be selected for use with an AAV capsid having a particular efficiency for targeting CNS or tissues or cells within the CNS. In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The expression cassette typically contains a promoter sequence as part of the expression control sequences, e.g., located between the selected 5′ ITR sequence and the immunoglobulin construct coding sequence. Tissue specific promoters, constitutive promoters, regulatable promoters [see, e.g., WO 2011/126808 and WO 2013/04943], or a promoter responsive to physiologic cues may be used may be utilized in the vectors described herein. In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product.

These control sequences are “operably linked” to the immunoglobulin construct gene sequences. As used herein, the term “operably linked” refers to both expression control sequences that are contiguous with the gene of interest and expression control sequences that act in trans or at a distance to control the gene of interest.

In one embodiment, a self-complementary AAV is provided. This viral vector may contain a Δ5′ ITR and an AAV3′ ITR. In another embodiment, a single-stranded AAV viral vector is provided. Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The available space for packaging may be conserved by combining more than one transcription unit into a single expression cassette, thus reducing the amount of required regulatory sequences. For example, a single promoter may direct expression of a single cDNA or RNA that encodes two or three or more genes, and translation of the downstream genes are driven by IRES sequences. In another example, a single promoter may direct expression of a cDNA or RNA that contains, in a single open reading frame (ORF), two or three or more genes separated from one another by sequences encoding a self-cleavage peptide (e.g., 2A) and/or a protease recognition site (e.g., furin). The ORF thus encodes a single polyprotein, which, either during or after translation, is cleaved into the individual proteins (such as, e.g., heavy chain and light chain). It should be noted, however, that although these IRES and polyprotein systems can be used to save AAV packaging space, they can only be used for expression of components that can be driven by the same promoter. In another alternative, the transgene capacity of AAV can be increased by providing AAV ITRs of two genomes that can anneal to form head to tail concatamers.

In the examples below, an AAV9 vector is described for expressing trastuzumab directly in the CNS to treat breast cancer CNS metastases. AAV9 vectors are described, e.g., in U.S. Pat. No. 7,906,111, which is incorporated herein by reference. However, other sources of AAV capsids and other viral elements may be selected, as may other immunoglobulin constructs and other vector elements. Methods of generating AAV vectors have been described extensively in the literature and patent documents, including, e.g., WO 2003/042397; WO 2005/033321, WO 2006/110689; U.S. Pat. No. 7,588,772 B2. The source of AAV capsids may be selected from an AAV which targets CNS, specific cells within the CNS, and/or specific cancer-associated antigens or receptors. Suitable AAV may include, e.g, AAV9 [U.S. Pat. No. 7,906,111; US 2011-0236353-A1], rh10 [WO 2003/042397] and/or hu37 [see, e.g., U.S. Pat. No. 7,906,111; US 2011-0236353-A1]. However, other AAV, including, e.g., AAV1, AAV2, AAV3, AAV4, AAV5, AAV6, AAV7, AAV8 [U.S. Pat. Nos. 7,790,449; 7,282,199] and others such as, e.g., those described in a word seems to be missing here may be selected for preparing the AAV vectors described herein.

### Uses and Regimens

Suitably, the composition of the invention are designed so that AAV vectors carry the nucleic acid expression cassettes encoding the immunoglobulin constructs and regulatory sequences which direct expression of the immunoglobulin thereof in the selected cell. Following administration of the vectors into the CNS, the vectors deliver the expression cassettes to the CNS and express the proteinaceous immunoglobulin constructs in vivo. The use of compositions described herein in an anti-neoplastic method are described, as are uses of these compositions in anti-neoplastic regimens, which may optionally involve delivery of one or more other anti-neoplastic or other active agents.

As stated above, a composition may contain a single type of AAV vector as described herein which contains the expression cassette for delivering the anti-neoplastic immunoglobulin construct in vivo. Alternatively, a composition may contain two or more different AAV vectors, each of which has packaged therein different expression cassettes. For example, the two or more different AAV may have different expression cassettes which express immunoglobulin polypeptides which assemble in vivo to form a single functional immunoglobulin construct. In another example, the two or more AAV may have different expression cassettes which express immunoglobulin polypeptides for different targets, e.g., two provide for two functional immunoglobulin constructs (e.g., an anti-Her2 immunoglobulin construct and a second anti-neoplastic immunoglobulin construct). In still another alternative, the two or more different AAV may express immunoglobulin constructs directed to the same target, wherein one of the immunoglobulin constructs has been modified to ablate FcRn binding and a second immunoglobulin construct which retains its ability or has enhanced ability to bind to FcRn. Such a composition may be useful to simultaneously provide antibodies with increased retention in the brain area and antibodies for systemic delivery of the immunoglobulin construct.

Optionally, one or both of these immunoglobulin constructs described herein has enhanced ADCC activity. A regimen as described herein may comprise, in addition to one or more of the combinations described herein, further combination with one or more of an anti-neoplastic biological drug, an anti-neoplastic small molecule drug, a chemotherapeutic agent, immune enhancers, radiation, surgery, and the like. A biological drug as described herein, is based on a peptide, polypeptide, protein, enzyme, nucleic acid molecule, vector (including viral vectors), or the like.

Suitably, the compositions described herein comprise an anti-neoplastic effective amount of one or more AAV suspended in a pharmaceutically suitable carrier designed for delivery to the subject via injection, osmotic pump, intrathecal catheter, or for delivery by another device or route. In one example, the composition is formulated for intrathecal delivery. As used herein, intrathecal delivery encompasses an injection into the spinal canal, more specifically into the subarachnoid space. However, other routes of delivery may be selected and the pharmaceutically acceptable carriers for the AAV compositions including, e.g., intracranial, intranasal, intracisternal, intracerebrospinal fluid delivery, among other suitable direct or systemic routes, i.e. Ommaya reservoir.

The compositions can be formulated in dosage units to contain an amount of AAV that is in the range of about 1×10⁹ genome copies (GC) to about 5×10¹³ GC (to treat an average subject of 70 kg in body weight). In one embodiment, a spinal tap is performed in which from about 15 mL (or less) to about 40 mL CSF is removed and in which vector is admixed with the CSF and/or suspended in a compatible carrier and delivered to the subject. In one example, the vector concentration is about 3×10¹³ GC, but other amounts such as about 1×10⁹ GC, about 5×10⁹ GC, about 1×10¹⁰ GC, about 5×10¹⁰ GC, about 1×10¹¹ GC, about 5×10¹¹ GC, about 1×10¹² GC, about 5×10¹² GC, or about 1.0×10¹³ GC.

The rAAV, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, maltose, and water. The selection of the carrier is not a limitation of the present invention. Optionally, the compositions of the invention may contain, in addition to the rAAV and carrier(s), other conventional pharmaceutical ingredients, such as preservatives, or chemical stabilizers.

In one embodiment, the compositions described herein are used in a method for retarding the growth of a tumor. In still another embodiment, the compositions described herein are useful for decreasing tumor size in a subject. In a further embodiment, the compositions described herein are useful in reducing the number of cancer cells in a non-solid tumor cancer. In another embodiment, a composition as provided herein is used in a method for increasing overall survival and/or progression-free survival in a patient. For example, the data in the Examples below demonstrates a 33% increase in survival rate in metastatic breast cancer in brain as a solo therapy over the period tested. However, even more modest increases in survival rate would be desirable. The anti-neoplastic immunoglobulin constructs are selected with a view to the neoplasm to be treated. For example, for treatment of a metastatic breast cancer in the brain, one may engineer an expression cassette for an anti-HER antibody into a recombinant AAV as described herein. Optionally, the AAV compositions as described herein are administered in the absence of an additional extrinsic pharmacological or chemical agent, or other physical disruption of the blood brain barrier.

In a combination therapy, the AAV-delivered immunoglobulin construct described herein is administered before, during, or after commencing therapy with another agent, as well as any combination thereof, i.e., before and during, before and after, during and after, or before, during and after commencing the anti-neoplastic therapy. For example, the AAV can be administered between 1 and 30 days, preferably 3 and 20 days, more preferably between 5 and 12 days before commencing radiation therapy. In another embodiment of the invention, chemotherapy is administered concurrently with or, more preferably, subsequent to AAV-mediated immunoglobulin (antibody) therapy. In still other embodiments, the compositions of the invention may be combined with other biologics, e.g., recombinant monoclonal antibody drugs, antibody-drug conjugates, or the like. Further, combinations of different AAV-delivered immunoglobulin constructs such as are discussed above may be used in such regimens.

Any suitable method or route can be used to administer an AAV-containing composition as described herein, and optionally, to co-administer anti-neoplastic agents and/or antagonists of other receptors. The anti-neoplastic agent regimens utilized according to the invention, include any regimen believed to be optimally suitable for the treatment of the patient's neoplastic condition. Different malignancies can require use of specific antitumor antibodies and specific anti-neoplastic agents, which will be determined on a patient to patient basis. Routes of administration include, for example, systemic, oral, intravenous, intraperitoneal, subcutaneous, or intramuscular administration. The dose of antagonist administered depends on numerous factors, including, for example, the type of antagonists, the type and severity tumor being treated and the route of administration of the antagonists.

The following examples are illustrative only and are not a limitation on the invention described herein.

### EXAMPLES

### Example 1: CNS Expression of AAV9-Mediated Delivery of GFP

Both GFP and mAbs have been expressed in the CNS of cynomolgus macaques following intracisternal injection of AAV9 vectors containing a GFP transgene under either CMV or CB7 promoters at 5×10¹² genome copies (gc)/kg. After 14 days, macaques were necropsided and histology and biodistribution studies were performed. Post-necropsy histological analysis showed broad CNS expression of GFP in cerebrum, cerebellum, choroid plexus, meninges, and spinal cord ventral horn.

In addition, 3×10¹² gc/kg of AAV9 vector containing the 201 anti-SIV immunoadhesin (201IA) transgene under the control of the CB7 promoter was injected intracisternally and CSF was taken at regular intervals to measure the concentration of the immunoadhesin. The resulting level of 201IA expressed in the CSF peaked at ˜600 ng/mL, plateaued at ˜250 ng/mL, and remained stable at 198 days post-injection.

### A. 201IA Expression Construct

The codon-optimized nucleotide sequence for rhesus macaque anti-SIV mac251 gp120 IgG-201 (Glamann et al. J Virol. 1998; 74(15):7158-7163. doi:10.1128/JVI. 74.15.7158-7163.2000.Updated.) immunoadhesin (201IA) was cloned into an AAV expression construct. The construct was flanked by AAV2 inverted terminal repeats and contained a CB7 promoter, a chimeric intron, and a rabbit globin polyadenylation sequence (pAAV.CB7.CI.201IA.rBG).

### B. I253A Mutation of 201 IA to Abrogate FcRn Binding

A nucleotide sequence 768 bp in length complementary to the 201IA gene but containing a mutation corresponding to I253A [SEQ ID NO: 24 provides the CH2.CH3 fragment with this mutation] or H453A [SEQ ID NO: 23 provides the CH2.CH3 fragment with this mutation] of the heavy chain amino acid sequence (Kabat numbering) was obtained from GeneArt (Life Technologies). The sequence was flanked by Pst1 and BstZ171 restriction sites matching those in pAAV.CB7.CI.201IA.rBG. The mutated sequences were separately cloned into a pAAV.CB7.CI.201IA.rBG by restriction digest using the enzymes indicated (NEB) and ligation (TaKaRa Inc.) as described by the manufacturers. Sanger sequencing (GeneWiz) was used to confirm complementarity of pAAV.CB7.CI.201IA.rBG [SEQ ID NO: 5 (SEQ ID NO: 6 corresponds to encoded 201IA sequence)], pAAV.CB7.CI.201IA(I253A).rBG [SEQ ID NO: 7 (encoding SEQ ID NO:8)] and pAAV.CB7.CI.201IA(H435A).rBG [SEQ ID NO: 9 (encoding SEQ ID NO:10] on either side of the desired mutation.

### B. IA Expression in HEK293 Cells and Purification by Protein A

3×10⁸ HEK293 cells (293 cells) were seeded in a 10-stack CellSTACK® (Corning) in DuLbecco's Modified Eagle's Medium (DMEM, Corning CellGro) supplemented with 10% FBS and 1% penicillin/streptomycin (DMEM complete) and incubated at 37° C. 5% CO₂ for 48 hours. 1 mg of pAAV.CB7.CI.201IA.rBG or pAAV.CB7.CI.201IA(I253A).rBG in TE buffer (Qiagen) was diluted in 42 mL room-temperature antibiotic and serum-free DMEM. 2 mL PEI-Max 40 KDa, linear (Polysciences) at 1 mg/mL and pH 7.1 was diluted separately in 42 mL room-temperature antibiotic and serum-free DMEM. Diluted DNA and diluted PEI were combined and incubated for 15 minutes at room temperature. The DNA-PEI mixture was added to 1 L final volume of antibiotic and serum-free DMEM. 293T cells were washed twice with sterile PBS. The DNA-PEI DMEM mixture was added and incubated with the cells for 72 hours at 37° C. 5% CO₂. Supernatant was harvested and centrifuged for 10 minutes at 3000 x g to pellet cellular debris. Supernatant was then concentrated using Centricon® Plus-70 Centrifugal Filter Units (EMD Millipore) according to manufacturer's instructions. 201IA or 201IA(I253A) was then purified using a Protein A Antibody Purification Kit (Sigma) and quantified using a NanoDrop 2000 (Thermo Scientific). The purified IAs were then diluted to 1 mg/mL using glycerol and stored at −20° C.

### C. SDS-PAGE/Western Blot Analysis of IAs

SDS-PAGE using NuPage reagents (Life Technologies) was performed according to the manufacturer's instructions. Briefly, 1 μg 201IA, 201IA(I253A), or 201IA(H453A) was purified from 293 supernatant or 201IA previously purified in-house was mixed with NuPage Sample Buffer and NuPage Reducing Agent and heated at 70° C. for 10 minutes. Precast NuPage 4-12% Bis-Tris 1 mm acrylamide gels were loaded with samples and MagicMark XP Western Protein Standard (LifeTechnologies) and run in 1× NuPage MOPS SDS Running Buffer at 200V for 1 hour. The Trans-Blot® Turbo™ lx transfer system (BioRad) was used to transfer proteins to LF PVDF membranes. Ion reservoir stacks were wetted with 1× Trans-Blot® Turbo™ (TBT) transfer buffer for 2-3 minutes. Pre-cut LF PVDF membranes were immersed in 100% ethanol until translucent, then transferred to 1×TBT buffer for 2-3 minutes. The transfer stack was assembled and run at 1.3 A and 25 V for 7 minutes. The LF PVDF membrane was blocked overnight with gentle shaking in 1×NET buffer+2% gelatin (50 mM Tris HCL pH 7, 125 mM NaCl, 5 mM EDTA pH 8, 0.05% Triton X-100, 2% gelatin in double distilled H2O). Goat anti-human IgG polyclonal antibody conjugated to biotin (Abcam) was diluted in 1×NET+2% gelatin and incubated with the membrane at room temperature, washed with 1×NET, incubated with streptavidin-horseradish peroxidase (Abcam) diluted in 1×NET+2% gelatin, and washed with 1×NET. The Western blot was detected using SuperSignal® West Pico Chemiluminescence Substrate (Thermo Scientific) according to the manufacturer's protocol. Images were captured using the BioRad ChemiDoc™ MP Image System (Thermo Scientific) with high resolution chemiluminescence automatic settings.

### D. 201IA ELISA

All procedures were conducted at room temperature unless indicated otherwise. Plates were washed with BioTek 405TS microplate washer using PBS+0.05% Tween-20. mac251 gp120 (Immune Technology Corp.) diluted to 2 μg/mL in PBS was incubated overnight on Costar® 96-well EasyWash™ ELISA assay plates (Corning) at 4° C. Plates were then blocked 201IA ELISA blocking buffer (PBS+5% heat-inactivated fetal bovine serum+1 mM EDTA+0.07% Tween-20). Diluted samples were added to plates and diluted 2-fold down the plate at least four times. Plates were incubated for 1 h at 37° C. and blocked again in 201IA ELISA blocking buffer. Plates were then incubated with AffiniPure polyclonal goat anti-human IgG-biotin (Jackson ImmunoResearch Labs) diluted in PBS then with streptavidin-horseradish peroxidase (Abcam) diluted in PBS. 3,3′,5,5′-tetramethylbenzidine (TMB) substrate was used to develop the plates. After stopping the colorimetric reaction with H₂SO₄, plates were read using a SpectraMax M3 (Molecular Devices) plate reader at 450 nm.

Equivalent performance of 201IA or 201IA(I253A), and performance of 201(H453) purified from 293 cells as described in these examples and a 201IA standard protein produced in-house was determined by 201IA ELISA. Each IA was diluted to 50 ng/mL and assayed as described above. The 201IA used as a standard was produced as follows. RAG KO mice were injected with an AAV8.TBG.201IA vector at 3×10¹¹ GC/per mouse intravenously, and orbital bleeds were collected on a weekly basis for 8 weeks, mice terminated by cardiac bleed. There are generally 5 mice per group. All collected serum is pooled together and loaded on the protein A affinity column from SIGMA as described above. Generally, purified 201IA is diluted to 1 mg/ml and glycerol added so that final glycerol is about 20% for better storage.

### E. AAV9 Vector Production

pAAV.CB7.CI.201IA.rBG and pAAV.CB7.CI.201IA(I253A).rBG were packaged in an AAV9 capsid by triple transfection of 293 cells and purified as previously described in M. Lock et al, Hum Gene Ther. 2010 October; 21(1); 1259-1271, published online 2010 Sep. 24.

### F. Expression of 201IA and 201 IA(I253A) in Brain and Serum of Mice

All animals were maintained according to NIH and USDA guidelines for the care and use of animals in research. 6-8 week-old female Rag1−/−(Jackson Labs #002216), FcRn−/− Rag1−/−(Jackson Labs #017700), or human FcRn transgenic mice (mFcRn−/−hFcRn+/+, Jackson Labs #016919) on a C57BL/6 background were obtained and kept at the University of Pennsylvania.

For vector administration, AAV9.CB7.CI.201IA.rBG or AAV9.CB7.CI.201IA(I253A).rBG was diluted in sterile PBS. For intravenous (IV) administration, vector was diluted to 1×10¹⁰ or 1×10¹¹ genome copies (GC) per 100 μL. For intracerebroventricular (ICV) administration, vector was diluted to 1×10¹⁰ GC or 1×10¹¹ GC per 10 μL. IV injection was performed by tail-vein injection, and ICV injection was performed free-hand as described previously (Glascock et al. J Vis Exp. 2011 Oct. 3; (56)) after isofluorane induction of anesthesia. Blood was taken at days 3, 7, 14, 21, 28, 42, 56, and a final time point of day 60 for 201IA(I253A) or day 76 for 201IA by retro-orbital bleed into Z-Gel™ microtube serum separators (Sarstedt). Blood was incubated at room temperature for 20 minutes then centrifuged for 5 minutes at 5000 x g. Serum was kept at −80° C. and used in the 201IA ELISA described above in Part D to measure serum 201IA concentration.

At necropsy, mice were deeply anesthetized with 100 mg/kg ketamine and 10 mg/kg xylazine in sterile PBS to a spinal plane of anesthesia. The thoracic cavity was exposed. A 20 gauge Angiocath™ Autoguard™ IV catheter (Becton Dickenson) was inserted into the left ventricle of the heart, and the right atrium was nicked with scissors. 50 mL PBS with Heparin (10 U/mL, Sigma) was administered into the left ventricle slowly through the IV catheter using a 30 mL hand-held syringe. Fluid exiting the right atrium was clear at the end of the perfusion procedure. Brain, liver, and spleen were removed and frozen immediately on dry ice. Brain tissue extract was prepared by quartering frozen mouse brains (˜100 mg brain per quarter) and immersing them in 1 mL tissue lysis buffer (25 mM Tris-HCl, 5 mM EDTA, 1% Triton™-X, 150 mM NaCl, pH 7.6). Samples were homogenized with stainless-steal beads using a TissueLyzer™ (Qiagen) at 30 Hz for 2 minutes, frozen overnight at −80° C., thawed in a room-temperature water bath, and centrifuged at 10K x g for 10 minutes at 4° C. Supernatants from each of the four sections of an individual mouse brain were combined. After gentle vortexing, the brain extracts were aliquoted and frozen at −80° C. until use. Diluted brain extracts were used in the 201IA ELISA described above to determine 201IA expression in brain.

Serum expression of each the I253A and the H435A 201IA mutants was significantly lower than the wild-type 201IA (standard) after both iv or icv administration at both tested doses (1×10¹⁰ GC/mouse or 1×10¹¹ GC/mouse). The brain extracts tested after ICV administration (1×10¹¹ GC/mouse) showed expression of the I253A mutant in the brain at levels exceeding those of the wild-type (standard). Expression of H435A (1×10¹¹ GC/mouse) was observed.

### G. Expression of 201 IA and 201 IA(I253A) in CSF and Serum of Cynomolgus Macaques

Four 3-4 year-old female cynomolgus macaques weighing between 3-5 kg were housed in stainless steel caging on a 12-hour light/dark cycle at the University of Pennsylvania according to according to NIH and USDA guidelines for the care and use of animals in research. Animals were acclimatized 7 weeks prior to initiation of studies. Monkeys were given Primate Diet 5049 (PMI Feeds Inc.). Water was given ad lib from an automatic watering system.

On the day of vector administration, animals were anesthetized using ketamine (10-15 mg/kg) and dexmedetomidine (0.05-0.10 mg/kg) given intramuscularly (IM). Animals were weighed, and vital signs were recorded. The hair over the back of the head and cervical spine was shaved. The skin was sterilely prepped with betadine. The neck was flexed so that the chin was almost touching the chest (care was taken not to occlude the animal's airway). The occipital protuberance at the back of the skull and the wings of the atlas (C1) were palpated and the spinal needle or regular needle (20-24 gauge) was inserted midway between them. If bone was encountered, the needle was redirected anteriorly or posteriorly. Once in the subarachnoid space, CSF was collected via gravitational flow into a syringe or other sterile container as it welled up into the hub of the needle (up to 2 mL). Suction was not applied to the needle. Up to 2 mL of vector solution was injected using a syringe pump at 0.5 mL/minute or manually at a slow steady pace. The needle was removed and direct pressure applied to the puncture site. Two macaques received AAV9.CB7.CI.201IA.rBG.N401 and two received AAV9.CB7.CI.201IA(I253A).rBG. The dose was 1.00×10¹² VG per kilogram of body weight.

At least once every two weeks, animals were monitored for vital signs, clinical pathology, and immunology. Blood and lumbar CSF were collected at day 8 and 15 after the procedure, then monthly. Serum and CSF was stored at approximately −65 to −80° C. 201IA expression was evaluated by 201IA ELISA as indicated above. Changes in the blood chemistries and blood profiles of the animals were monitored by the contract facility Antech Diagnostics, Inc.

Monkeys will be euthanized at the end of their study period. The animals are first sedated with ketamine (10-15 mg/kg) and dexmedetomidine (0.05-0.10 mg/kg) IM. They are euthanized using sodium pentobarbital (80 mg/kg IV). Death is confirmed by absence of heartbeat and respiration. The animal may also be exsanguinated to assure death. Collected tissues will be placed in 10% neutral buffered formalin for histopathology. For genome copy analysis, tissue samples will be immediately frozen on dry ice and maintained at <−60° C. Samples will be directly frozen in OCT embedding medium for cryosectioning. Slides will be prepared by Cellular and Morphology Core of the Gene Therapy Program of the University of Pennsylvania. Other appropriate stains may be employed at the discretion of the study pathologist.

### Example 2: Production of AAV9 Expressing Trastuzumab

A well-published murine xenograft model of breast cancer brain metastasis is used to determine if trastuzumab expressed in the CNS prolongs survival or alleviates tumor burden [Martinez-Aranda A, et al, Development of a Preclinical Therapeutic Model of Human Brain Metastasis with Chemoradiotherapy. Int J Mol Sci. 2013; 14:8306-8327]. HER2 positive human BT474 ductal carcinoma cells are transfected with luciferase and injected stereotactically into the brain parenchyma of nude mice. Tumor size will be monitored by luminescent intensity. When the tumors grow to 10 mm², the mice will be injected intraventricularly with varying concentrations of AAV9 vector carrying a trastuzumab transgene.

The transgene is created by cloning the codon-optimized nucleic acid sequences now provided in SEQ ID NO: 1, which encode the published sequences of the light and heavy variable chains of trastuzumab, into an IgG expression cassette. The constant region amino acid sequences described in WO 2015/012924, which is incorporated by reference herein, can be used. See, e.g., Carter P, et al, Humanization of an anti-p185HER2 antibody for human cancer therapy. Proc Natl Acad Sci USA. 1992 May 15; 89(10):4285-9 describing humanization of the murine mAb precursor of trastuzumab. These amino acid sequences exactly match those of the clinical product sequenced by mass spectrometry in 2013 [Gahoual R, et al, Rapid and multi-level characterization of trastuzumab using sheathless capillary electrophoresis-tandem mass spectrometry. MAbs. 2013 Apr. 5; 5(3). [Epub ahead of print]. After injection of the vector, tumor size and mouse survival will be monitored for 30 days. At necropsy pathological examination of the tumor will be conducted and level of trastuzumab expression by ELISA of brain extracts determined. The vector and method described herein should provide prolonged survival, progression-free survival, and/or regression and/or stabilization of tumor burden.

### A. Trastuzumab Expression Construct

Sequences matching the WHO published sequences of the heavy and light chains of trastuzumab were obtained from GeneArt (Life Technologies). The light chain sequence was flanked by EcoRV and BsiW1 restriction sites, and the heavy chain sequence was flanked by Xba1 and Sal1 restriction sites [the nucleic acid sequence is provided in SEQ ID NO: 11, which encodes trastuzumab heavy chain variable (SEQ ID NO: 12), heavy chain constant (SEQ ID NO: 13), light chain variable (SEQ ID NO: 14), kappa chain (SEQ ID NO: 15), and Amp-R (SEQ ID NO: 16)] provides the sequences of the plasmids containing the trastuzumab heavy and light chains.

The heavy and light chain sequences were cloned into an AAV expression construct using restriction digest (NEB) and ligation (TaKaRa Inc.) using known cloning techniques. The heavy and light chain sequences were separated from each other by an F2A self-cleaving peptide. The construct was flanked by AAV2 inverted terminal repeats and contained a CMV immediate early promoter, a chimeric intron, and a SV40 polyadenylation signal, termed pAAV.CMV.CI.trastuzumab.SV40 [SEQ ID NO: 17, encoding trastuzumab heavy chain variable, heavy chain constant, light chain variable, kappa chain (SEQ ID NO: 18-21, respectively)].

### B. Trastuzumab Expression in HEK293 Cells and Purification by Protein A

3×10⁸ HEK293 cells [obtained from the ATCC] were seeded in a 10-stack CellSTACK® in DMEM complete at 37° C. 5% CO₂ for 48 hours. 1 mg of pAAV.CMV.CI.trastuzumab.SV40 (described in Part A) was diluted in 42 mL room-temperature antibiotic and serum-free DMEM. 2 mL PEI-Max 40 KDa, linear (Polysciences) at 1 mg/mL and pH 7.1 was diluted separately in 42 mL room-temperature antibiotic and serum-free DMEM. Diluted DNA and diluted PEI were combined and incubated for 15 minutes at room temperature. The DNA-PEI mixture was added to a 1 L final volume of antibiotic and serum-free DMEM. 293 cells were washed twice sterile PBS, and the cells were then incubated with the DNA-PEI DMEM mixture for 72 hours. Supernatant was harvested, centrifuged for 10 minutes at 3000 x g to pellet cellular debris, and concentrated using Centricon® Plus-70 Centrifugal Filter Units (EMD Millipore) according to manufacturer's instructions. Trastuzumab was then purified using a Protein A Antibody Purification Kit (Sigma) and quantified using a NanoDrop 2000 (Thermo Scientific). The purified trastuzumab was diluted to 1 mg/mL using glycerol and stored at −20° C.

### C. SDS-PAGE/Western Blot Analysis of IAs

SDS-PAGE using NuPage reagents (Life Technologies) was performed according to the manufacturer's instructions. Briefly, 1 μg trastuzumab purified from 293 supernatant as described in Part B of this Example or trastuzumab clinical product resuspended in PBS (Hoffmann-La Roche, HUP Pharmacy), was mixed with NuPage Sample Buffer and NuPage Reducing Agent and heated at 70° C. for 10 minutes. Precast NuPAGE® 4-12% gradient Bis-Tris (neutral pH) 1 mm acrylamide gels were loaded with samples and MagicMark™ XP Western Protein Standard (LifeTechnologies) and run in 1× NuPage® 3-morpholinopropane-1-sulfonic acid (MOPS) SDS Running Buffer at 200V for 1 hour. The Trans-Blot® Turbo™ 1× transfer system (BioRad) was used to transfer proteins to low fluorescence (LF) polyvinylidene fluoride (PVDF) membranes. Ion reservoir stacks were wetted with 1× Trans-Blot® Turbo™ (TBT) transfer buffer for 2-3 minutes. Pre-cut LF PVDF membranes were immersed in 100% ethanol until translucent, then transferred to 1× TBT buffer for 2-3 minutes. The transfer stack was assembled and run at 1.3 A and 25 V for 7 minutes. The LF PVDF membrane was blocked overnight with gentle shaking in 1×NET buffer+2% gelatin (50 mM Tris HCl pH 7, 125 mM NaCl, 5 mM ethylenediaminetetraacetic acid (EDTA) pH 8, 0.05% Triton™ X-100 (Triton™ X-100 is a nonionic surfactant that has a hydrophilic polyethylene oxide chain and an aromatic hydrocarbon lipophilic or hydrophobic group), 2% gelatin in double distilled H2O). Goat anti-human IgG polyclonal antibody conjugated to biotin (Abcam) was diluted in 1×NET+2% gelatin and incubated with the membrane at room temperature, washed with 1×NET, incubated with streptavidin-horseradish peroxidase (Abcam) diluted in 1×NET+2% gelatin, and washed with 1×NET. The Western blot was detected using SuperSignal® West Pico Chemiluminescence Substrate (Thermo Scientific) according to the manufacturer's protocol. Images were captured using the BioRad ChemiDoc™ MP Image System (Thermo Scientific) with high resolution chemiluminescence automatic settings.

### D. Vector Production

pAAV.CMV.CI.trastuzumab.SV40 was packaged in an AAV9 capsid by triple transfection of 293 cells and purified as previously described (Lock et al, 2010, cited above).

### E. Trastuzumab ELISA

A trastuzumab mimotope ELISA was developed as described previously (Jiang et al. J Biol Chem. 2005 Feb. 11; 280(6):4656-62. Epub 2004 Nov. 9). All steps were performed at room temperature unless otherwise stated. Plates were washed with a BioTek 405TS microplate washer. A peptide mimotope of the epitope of HER2 to which trastuzumab binds, LLGPYELWELSH [SEQ ID NO: 22], was obtained from the mimotopes, resuspended in DMSO, and stored at −80° C. Costar® 96-well EasyWash™ ELISA assay plates (Corning) were coated at 1 μg/mL LLGPYELWELSH [SEQ ID NO: 22] in 100 mM bicarbonate solution (pH 9.6), incubated overnight at 4° C., and blocked with trastuzumab ELISA blocking buffer (TEB, PBS+5% bovine serum albumin+1 mM EDTA+0.07% Tween-20). Samples were diluted in TEB and plated, diluted 2-fold down the ELISA plate in TEB, and incubated. Plates were then incubated with AffiniPure polyclonal goat anti-human IgG-biotin (Jackson ImmunoResearch Labs) diluted in TEB followed by streptavidin-horseradish peroxidase (Abcam) diluted in TEB. Plates were developed with TMB substrate, stopped with 2N H₂SO₄, then read using a SpectraMax M3 (Molecular Devices) plate reader at 450 nm.

### F. Expression of 201 IA and 201 IA(I253A) in Brain and Serum of Mice

6-8 week-old female Rag1−/− mice (Jackson Labs #002216) on a C57BL/6 background were obtained and kept at the University of Pennsylvania and maintained according to NIH and USDA guidelines for the care and use of animals in research.

For vector administration, pAAV.CMV.CI.trastuzumab.SV40 was diluted in sterile PBS. For intravenous (IV) administration, vector was diluted to 1×10¹⁰ or 1×10¹¹ GG per 100 μL. For intracerebroventricular (ICV) administration, vector was diluted to 1×10¹⁰ or 1×10¹¹ GC per 10 μL. IV injection was performed by tail-vein injection, and ICV injection was performed free-hand after isofluorane induction of anesthesia as described previously (Glascock et al.). Blood was taken at days 3, 7, 14, 21, 28, 42, 56, and 60 post-vector administration by retro-orbital bleed into Z-Gel microtube serum separators (Sarstedt). Blood was incubated at room temperature for 20 minutes then centrifuged for 5 minutes at 5000 x g. Serum was kept at −80° C. and used in the trastuzumab ELISA described above to measure serum trastuzumab concentration.

At necropsy, mice were deeply anesthetized with 100 mg/kg ketamine and 10 mg/kg xylazine in sterile PBS to a spinal plane of anesthesia. The thoracic cavity was exposed. A 20 gauge Angiocath™ Autoguard™ IV catheter (Becton Dickenson) was inserted into the left ventricle of the heart, and the right atrium was nicked with scissors. 50 mL PBS with Heparin (10 U/mL, Sigma) was administered into the left ventricle slowly through the IV catheter using a 30 mL hand-held syringe. Fluid exiting the right atrium was clear at the end of the perfusion procedure. Brain, liver, and spleen were removed and frozen immediately on dry ice.

Brain tissue extract was prepared by quartering frozen mouse brains (˜100 mg brain per quarter) and immersing them in 1 mL tissue lysis buffer (25 mM Tris-HCl, 5 mM EDTA, 1% Triton-X, 150 mM NaCl, pH 7.6). Samples were homogenized with stainless-steal beads on a TissueLyzer (Qiagen) at 30 Hz for 2 minutes, frozen overnight at −80° C., thawed in a room-temperature water bath, and centrifuged at 10K x g for 10 minutes at 4 C. Supernatants from each of the four sections of a single mouse brain were combined. After gentle vortexing, the brain extract was aliquotted and frozen at −80° C. until use. Diluted brain extracts were used in the trastuzumab ELISA above to measure brain trastuzumab concentration.

These data show the steady state expression levels of >1000 μg/mL in the serum of Rag1−/− mice following intravenous vector delivery for the duration of the experiment (60 days). For the mice receiving ICV vector administration, the steady state expression level of >750 μg/mL is observed for the duration of the experiment (60 days). These amounts are believed to demonstrate expression of levels which will provide a therapeutic effect.

The brain studies revealed concentrations of between about 1200 to about 1800 μg trastuzumab in the test mice for those receiving 1×10¹⁰ and 1×10¹¹ intravenous vector. There did not appear to be any significant difference between these dosage levels for iv delivery. At the same doses, greater variation was observed for the vectors delivered at these concentrations via ICV. The concentrations varied from about 1000 μg to about 2500 μg.

### G. Generation of HER2+BT474-M1 Breast Cancer Cell Line Expressing Firefly Luciferase

HER2+BT474.M1 human ductal carcinoma cells at passage 27 were a generous gift from Louis Chodosh and Jason Ruth. [BT474.MI cells is a subclone of BT474 that can be obtained from California Pacific Medical Center; Si Tuen Lee-Hoeflich, et al., Cancer Res Jul. 15, 2008 68; 5878.] Cells were grown in a T75 tissue culture flask (Corning) in DMEM/F12 media (Corning Cellgro) supplemented with 10% FBS and 1% penicillin/streptomycin (DMEM/F12 complete). VSVG.HIV.SIN.cPPT.CMV.ff-luciferase.WPRE lentiviral vector was obtained from the Penn Vector Core [E. Coprini et al, Viruses, August 2010, 2(8): 1577-1588.] When BT474-M1 cells were 60-70% confluent, media was aspirated, cells were washed with sterile PBS, trypsinized, and counted. 2.5×10⁵ cells in 2 mL DMEM/F12 complete were added to the wells of a six-well tissue culture treated plate (Falcon) and incubated overnight at 37° C. 5% CO₂. Vector was diluted in antibiotic and serum free DMEM/F12 to 3.5×10⁸ VG/mL, and five 2-fold serial dilutions were prepared. 1 mL of the six vector dilutions were added to corresponding wells of the 6-well plate containing PBS-washed BT474-M1 cells and 1 mL antibiotic and serum-free DMEM/F12. The plate was incubated for 48 hours at 37° C. 5% CO₂. Media was aspirated and replaced with DMEM/F12 complete. No cytopathology was noted by microscopy. After another 72 hours, the cells in the 3 wells that received the highest concentration of vector were washed with sterile PBS, trypsinized, mixed, and cultured in a T75 flask in DMEM/F12 complete. After 72 hours at 37° C. 5% CO₂, cells were trypsinized and diluted to a concentration of 1 cell per 200 μL DMEM/F12 complete. 200 μL cell suspension per well was plated in a 96-well tissue culture-treated plate (Falcon) and incubated at 37° C. 5% CO₂ for 6 weeks. Media was changed every 2 weeks. Two weeks after plating, wells with single colonies of clonal cells were noted by microscopy. When wells were 70% confluent with a single cluster of clonal cells, fifteen clones were selected for further expansion to 70-80% confluency in 24-well plates, then in 6-well plates, then T25 tissue culture flasks (Corning). Morphology of the cells was compared with the parental BT474.M1 cell line and noted to be equivalent.

Luciferase activity of the cells was measured using the Dual Luciferase® Reporter Assay System (Promega) to the manufacturer's instructions. DNA was isolated from cells using the DNeasy Kit (Qiagen). Copy number of luciferase per cell in the five clones with the highest luminescence was determined by TaqMan Real Time PCR (Life Technologies) using the lentiviral packaging signal as a probe [A Hachiya et al, Gene Ther, April 2007; 14(8): 648-656, Epub 2007 Feb. 1]. The clone chosen for xenograft experiments was expanded to passage number 52 and cryopreserved in liquid nitrogen in DMEM/F12 with 5% DMSO and 20% FBS.

### H. Xenograft Model of HER2+ Breast Cancer Brain Metastases in Rag1−/− Mice

BT474-M1.ffluc cells prepared as described in Part G of this example were thawed, washed in DMEM/F12 complete, expanded in T175 flasks (Corning) at 37° C. 5% CO₂ in DMEM/F12 complete, and passaged once at least 1 week before tumor cell implantation. On the day of injection, cells at passage 53 were trypsinized at 70%-80% confluency and counted using a hemacytometer. Cells were centrifuged for 3 minutes at 1000 x g and washed with sterile PBS. After centrifuging again, cells were resuspended at 1×10⁵ cells/1 μL in sterile PBS and kept on ice until injection. For the tumor cell injection procedure, mice were anesthetized by intraperitoneal (IP) injection of 100 mg/kg ketamine and 10 mg/kg xylazine in sterile PBS to induce a spinal plane of anesthesia. Ophthalmic ointment was applied to the eyes of the mice ad lib. Hair was sheared from the top of the mouse's head using electric clippers. Estrogen pellets (1.6 mg, 60-day release) were implanted subcutaneously by cleansing the exposed skin first with povidone iodine then 70% ethanol. A small incision in the skin overlaying the thoracic spine was made, and the skin and underlying fascia were bluntly dissected. The estrogen pellet was implanted subcutaneously, and the incision was sutured with 4.0 vicryl. Next, the mice were then fixed in a stereotactic apparatus. The exposed skin over the skull was cleaned with povidone iodine followed by 70% ethanol. An anterior-posterior incision approximately 1 cm long was made over the top of the skull with a 22 scalpel blade. Bregma was identified. A pneumatic drill was positioned at bregma and coordinates were noted. The drill point was moved −0.8 mm anterior-posterior, +2.2 mm mediolateral of bregma, and a burr hole was drilled until brain parenchyma was reached. The drill was removed from the stereotactic apparatus, and a 10 μL Hamilton syringe was loaded with 1 μL of cell suspension. The needle was positioned on the apparatus, brought to bregma, and moved to the coordinates indicated above. The needle was checked for exact positioning over the burr hole, and coordinates were adjusted accordingly before penetrating −4.0 mm DV of bregma, then +1.0 mm. 1 μL of cell suspension was injected over 5 minutes. The needle was left in place for 5 minutes after injection, then removed slowly. The mouse was removed from the stereotactic apparatus, and 4.0 vicryl was used to suture the incision over the skull. Mice were placed in a clean cage on top of a heating pad set to 37° C. After recovering from anesthesia, the mice were given 100 μL of 15 mg/kg enrofloxacin (Bayer) in sterile PBS along with 0.3 mg/kg buprenorphine in sterile PBS subcutaneously. Mice received enrofloxacin subcutaneously for two days after the procedure.

Growth of tumor was monitored every 3-4 days using bioluminescent imaging (BLI). Mice were injected IP first with 150 mg/kg luciferin in sterile PBS then with 100 mg/kg ketamine and 10 mg/kg xylazine in PBS five minutes later. 5-10 minutes after anesthesia was administered, mice were imaged using an IVIS Xenogen imaging system. Bioluminescence was measured for at least 5 seconds. Regions of interest (ROI) corresponding to luminescent tumors were measured by drawing a gate around the ROI. Luminescence was reported in photons/second. At necropsy, mice were euthanized by overexposure to CO₂ followed by cervical dislocation. Brains were removed and preserved in formalin followed by 70% ethanol and embedded in paraffin for sectioning. Hematoxylin and eosin as well as luciferin immunostaining was performed. Liver and spleen were also taken at necropsy for biodistribution analysis of vector genomes.

### I. Prophylactic Treatment of Xenograft Model of Breast Cancer Brain Metastases

6-8 week-old female Rag1−/− mice (Jackson Labs #002216) were treated 21 days prior to tumor implantation with an ICV injection of 1×10¹¹ VG of AAV9.CMV.CI.trastuzumab.SV40 (n=9), AAV9.CB7.CI.201IA.rBG (n=10), or no treatment (n=5). Tumors were implanted and bioluminescence was measured as indicated above. Blood was taken retro-orbitally from mice on D20, 36, 62, and 72 post vector injection to measure serum trastuzumab as a surrogate for CNS trastuzumab expression. After reaching a tumor BLI of 1×10⁸ photons/second, mice are monitored daily and sacrificed at a clinical endpoint defined as neurological impairment or significant morbidity including lethargy, hunching, paralysis, neurological deficits, or seizures.

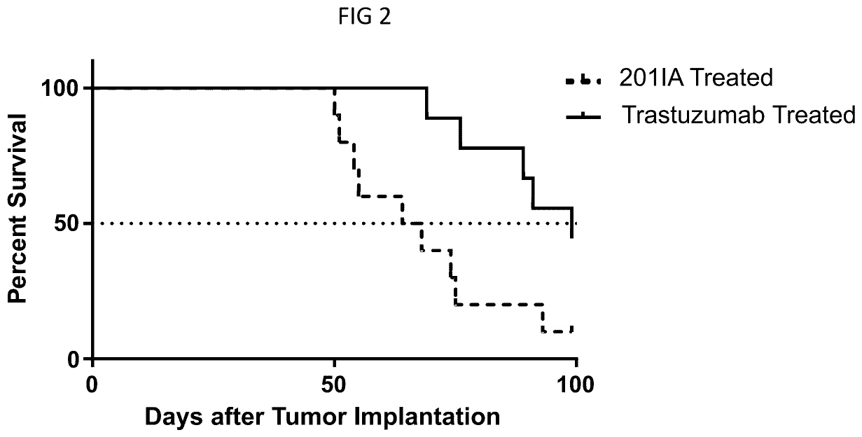

FIG. 2 provides a survival curve of mice given 1×10¹¹ GC ICV of AAV9.trastuzumab or AAV9.201IA prophylactically, then implanted with BT474-M1.ffluc tumor cells in the brain 21 days after vector administration. This curve reflects results 99 days post-tumor implantation. The median survival of 201IA group (sham treatment) is shown to be 66 days, whereas the median survival of the group treated with AAV9.trastuzumab is 99 days, a 33% increase in survival rate.

The biodistribution of the AAV9.trastuzumab as delivered iv and icv in Rag1−/− mice was assessed. At a dose of 1×10¹⁰ GC for both iv and icv-delivered vectors, relatively low levels of vector genomes are observed in either liver or brain. At the higher dose (1×10¹¹ GC), significantly higher levels of vectors are found in liver for both delivery methods, whereas significantly higher levels in brain are found only in the animals receiving icv administration. It is notable that this vector contained a non-tissue specific promoter. Safety concerns may be reduced via use of a tissue specific promoter which specifically targets cells of the brain and optionally other neural cells or cells in the central nervous system, in order to minimize expression in liver. Alternatively, expression in liver may be beneficial for the systemic delivery of trastuzumab in order to prevent or control breast cancer metastasis into other organs.

### J. Alternative Mouse Model Suitable for Studies of BT474-M1 Breast Cancer Brain Metastases Using NSG Mice

6-8 week-old female NSG mice (Jackson Labs #005557) were kept at the University of Pennsylvania. Tumors were implanted as indicated above with the following changes to the preparation of tumor cells and injection technique. Tumor cells were resuspended in 50% MatriGel® (Corning)/50% sterile PBS at 1×10⁵ cells per 5 μL. Injection volume was increased from 1 μL to 5 μL. After positioning the needle in the brain parenchyma, 5 minutes elapsed before injection began. The injection was performed slowly over 10 minutes, and the syringe was left in place 5 minutes before removal. Mice were monitored and bioluminescence was measured as indicated elsewhere in this document. The data showed successful engraftment of the tumor cells into brain. The rates of the tumor growth will be evaluated to determine if this model is desirable for study of breast cancer metastasis to brain.

### SEQUENCE LISTING FREE TEXT

the following information is provided for sequences containing free text under numberic idenifir <223>.

| SEQ ID NO: | |
|---|---|
| (containing | |
| free text) | Free text under <223> |
| 1 | <223> engineered anti-HER antibody |
| | <220> |
| | <221> misc |
| | <222> (1) . . . (60) |
| | <223> IL2 signal/leader peptide |
| | <220> |
| | <221> CDS |
| | <222> (61) . . . (423) |
| | <223> heavy variable |
| | <220> |
| | <221> Misc |
| | <222> (439) . . . (714) |
| | <223> CH1 |
| | <220> |
| | <221> Misc |
| | <222> (715) . . . (1410) |
| | <223> CH1 |
| | <220> |
| | <221> Misc |
| | <222> (2010) . . . (2069) |
| | <223> IL2 signal/leader peptide |
| | <220> |
| | <221> Misc |
| | <222> (2070) . . . (2391) |
| | <223> variable light |
| | <220> |
| | <221> Misc |
| | <222> (2407) . . . (2711) |
| | <223> light chain constant region |
| 2 | <223> Synthetic Construct |
| 3 | <223> engineered anti-HER heavy chain |
| | <220> |
| | <221> MISC_FEATURE |
| | <222> (1) . . . (20) |
| | <223> heavy chain signal peptide |
| | <220> |
| | <221> MISC_FEATURE |
| | <222> (21) . . . (140) |
| | <223> heavy chain variable region |
| | <220> |
| | <221> MISC_FEATURE |
| | <222> (141) . . . (259) |
| | <223> heavy chain constant region 1 |
| | <220> |
| | <221> MISC_FEATURE |
| | <222> (260) . . . (470) |
| | <223> heavy chain FC |
| 4 | <223> Engineered anti-Her2 light chain |
| | <220> |
| | <221> MISC_FEATURE |
| | <222> (1) . . . (20) |
| | <223> signal sequence |
| | <220> |
| | <221> MISC_FEATURE |
| | <222> (21) . . . (130) |
| | <223> light chain variable region |
| | <220> |
| | <221> MISC_FEATURE |
| | <222> (131) . . . (214) |
| | <223> light chain constant region |
| 5 | <223> pAAV.CB7.CI.201IA.rBG |
| | <220> |
| | <221> misc_feature |
| | <222> (275) . . . (404) |
| | <223> 3′ ITR (complement) |
| | <220> |
| | <221> misc_feature |
| | <222> (3226) . . . (3355) |
| | <223> 5′ ITR |
| | <220> |
| | <221> misc |
| | <222> (3423) . . . (3804) |
| | <223> CMV IE promoter |
| | <220> |
| | <221> CDS |
| | <222> (5161) . . . (6690) |
| | <223> 201IA |
| 6 | <223> Synthetic Construct |
| 7 | <223> Plasmid encoding 201IA(I253A)mutant |
| | <220> |
| | <221> misc |
| | <222> (1) . . . (130) |
| | <223> 5′ ITR |
| | <220> |
| | <221> misc_feature |
| | <222> (198) . . . (579) |
| | <223> CMV IE promoter |
| | <220> |
| | <221> promoter |
| | <222> (582) . . . (863) |
| | <223> CB promoter |
| | <220> |
| | <221> Intron |
| | <222> (958) . . . (1930) |
| | <223> chicken beta-actin intron |
| | <220> |
| | <221> CDS |
| | <222> (1936) . . . (3465) |
| | <223> CMV IE promoter |
| | <220> |
| | <221> polyA_signal |
| | <222> (3529) . . . (3655) |
| | <223> rabbit globin polyA |
| | <220> |
| | <221> misc_feature |
| | <222> (3744) . . . (3873) |
| | <223> 3′ ITR (complement) |
| | <220> |
| | <221> misc_feature |
| | <222> (4636) . . . (5493) |
| | <223> AP(R) marker |
| 8 | <223> Synthetic Construct |
| 9 | <223> engineered plasmid containing 201IA(H435) mutant |
| | <220> |
| | <221> misc_feature |
| | <222> (275) . . . (404) |
| | <223> 3′ ITR (complement) |
| | <220> |
| | <221> misc_feature |
| | <222> (3423) . . . (3804) |
| | <223> CMV IE promoter |
| | <220> |
| | <221> promoter |
| | <222> (3807) . . . (4088) |
| | <223> CB promoter |
| | <220> |
| | <221> TATA_signal |
| | <222> (4061) . . . (4064) |
| | <223> rabbit globin polyA |
| | <220> |
| | <221> Intron |
| | <222> (4183) . . . (5155) |
| | <223> chicken beta-actin intron |
| | <220> |
| | <221> CDS |
| | <222> (5161) . . . (6690) |
| | <223> CMV IE promoter |
| 10 | <223> Synthetic Construct |
| 11 | <223> Plasmid containing the heavy and light |
| | chains of trastuzumab |
| | <220> |
| | <221> misc_feature |
| | <222> (1) . . . (130) |
| | <223> 5′ ITR |
| | <220> |
| | <221> promoter |
| | <222> (191) . . . (932) |
| | <223> human CMV IE enhancer and promoter |
| | <220> |
| | <221> sig_peptide |
| | <222> (1305) . . . (1364) |
| | <223> IL-2 signal peptide |
| | <220> |
| | <221> CDS |
| | <222> (1365) . . . (1724) |
| | <223> Trastuzumab heavy variable |
| | <220> |
| | <221> CDS |
| | <222> (1725) . . . (2720) |
| | <223> trastuzumab heavy constant |
| | <220> |
| | <221> enhancer |
| | <222> (2726) . . . (3313) |
| | <223> IRES |
| | <220> |
| | <221> sig_peptide |
| | <222> (3314) . . . (3373) |
| | <223> IL2 signal peptide |
| | <220> |
| | <221> CDS |
| | <222> (3374) . . . (3694) |
| | <223> trastuzumab light variable |
| | <223> trastuzumab constant light (kappa) |
| | <220> |
| | <221> polyA_signal |
| | <222> (4038) . . . (4269) |
| | <223> SV40 late polyA signal |
| | <220> |
| | <221> polyA_signal |
| | <222> (4083) . . . (4269) |
| | <223> SV40 late polyA signal |
| | <220> |
| | <221> misc_feature |
| | <222> (4334) . . . (4463) |
| | <223> 3′ ITR (complement) |
| | <220> |
| | <221> rep_origin |
| | <222> (4640) . . . (5095)) |
| | <220> |
| | <221> CDS |
| | <222> (5226) . . . (6083) |
| | <223> amp-R |
| | <220> |
| | <221> misc_feature |
| | <222> (6257) . . . (6845) |
| | <223> COL/E1/origin |
| 12 | <223> Synthetic Construct |
| 13 | <223> Synthetic Construct |
| 14 | <223> Synthetic Construct |
| 15 | <223> Synthetic Construct |
| 16 | <223> Synthetic Construct |
| 17 | 223> Engineered plasmid |
| | containing trastuzumab MAb |
| | <220> |
| | <221> sig_peptide |
| | <222> (1254) . . . (1313) |
| | <223> IL2 signal sequence |
| | <220> |
| | <221> CDS |
| | <222> (1314) . . . (1673) |
| | <223> Trastuzamab heavy variable |
| | <220> |
| | <221> CDS |
| | <222> (1674) . . . (2660) |
| | <223> Trastuzamab heavy constant |
| | <220> |
| | <221> sig_peptide |
| | <222> (2745) . . . (2804) |
| | <223> IL2 signal sequence |
| | <220> |
| | <221> CDS |
| | <222> (2805) . . . (3110) |
| | <223> Trastuzamab light variable |
| | <220> |
| | <221> CDS |
| | <222> (3111) . . . (3452) |
| | <223> Trastuzamab light constant (kappa) |
| 18 | <223> Synthetic Construct |
| 19 | <223> Synthetic Construct |
| 20 | <223> Synthetic Construct |
| 21 | <223> Synthetic Construct |
| 22 | <223> Peptide mimotope of HER2 epitope |
| 23 | <223> trastuzumab immunoglobulin H435A mutant |
| 24 | <223> immunoglobulin fragment with I253A mutant |
| 25 | <223> Trastruzumab heavy chain |

U.S. patent application Ser. No. 16/986,946, filed Aug. 6, 2020, allowed, U.S. application Ser. No. 15/306,413, filed Oct. 24, 2016, now U.S. Pat. No. 10,780,182, which is a 371 of PCT/US15/27491, filed Apr. 24, 2015, now expired which claimed the benefit of U.S. Provisional Patent Application No. 61/984,646, filed Apr. 25, 20145, now expired, and all publications, patents, and patent applications cited in this application are hereby incorporated by reference in their entireties as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

1. (canceled)

2. A recombinant nucleic acid molecule comprising a vector genome comprising an AAV5′ inverted terminal repeat (ITR), an expression cassette, and an AAV3′ ITR,
wherein the expression cassette comprises:
(i) a nucleic acid coding sequence for an anti-Her2 immunoglobulin heavy chain having an exogenous IL-2 leader peptide, a heavy chain variable region having an amino acid sequence of SEQ ID NO: 18 and a heavy chain constant region,
(ii) a linker sequence,
(iii) a nucleic acid coding sequence for an anti-Her2 immunoglobulin light chain having an exogenous IL-2 leader peptide, a light chain variable region having an amino acid sequence of SEQ ID NO: 20, and a light chain constant region, and
(iv) regulatory sequences operably linked to the heavy chain and the light chain.

3. The recombinant nucleic acid molecule according to claim 2, wherein the coding sequence for the anti-Her2 immunoglobulin heavy chain comprises nucleic acid sequence encoding a heavy chain variable region comprising amino acid sequence of SEQ ID NO: 18, and a heavy chain constant region comprising amino acid sequence of SEQ ID NO: 19.

4. The recombinant nucleic acid molecule according to claim 2, wherein the coding sequence for the anti-Her2 immunoglobulin light chain comprises a nucleic acid sequence encoding a light chain variable region comprising amino acid sequence of SEQ ID NO: 20, and a light chain constant region comprising amino acid sequence of SEQ ID NO: 21.

5. The recombinant nucleic acid molecule according to claim 2, wherein the coding sequence for the anti-Her2 immunoglobulin heavy chain comprises a heavy chain variable region comprising nucleotides 1314 to 1673 of SEQ ID NO: 17 which encode the amino acid sequence of SEQ ID NO: 18, and a heavy chain constant region comprising nucleotides 1674 to 2660 of SEQ ID NO: 17 which encode the amino acid sequence of SEQ ID NO: 19.

6. The recombinant nucleic acid molecule according to claim 2, wherein the coding sequence for the anti-Her2 immunoglobulin light chain comprises a light chain variable region comprising nucleotides 2805 to 3110 of SEQ ID NO: 17 which encode the amino acid sequence of SEQ ID NO: 20, and a light chain constant region comprising nucleotides 3111 to 3452 of SEQ ID NO: 17 which encode the amino acid sequence of SEQ ID NO: 21.

7. The recombinant nucleic acid molecule according to claim 2, wherein the expression cassette comprises the coding sequence for the anti-Her2 immunoglobulin heavy chain comprising nucleotides 1314 to 2660 of SEQ ID NO: 17, and the coding sequence for the anti-Her2 immunoglobulin light chain comprising nucleotides 2805 to 3452 of SEQ ID NO: 17.

8. The recombinant nucleic acid molecule according to claim 2, wherein the expression cassette comprises the coding sequence for an anti-Her2 immunoglobulin heavy chain having an IL-2 leader peptide consisting of the nucleotides 1254 to 2660 of SEQ ID NO: 17.

9. The recombinant nucleic acid molecule according to claim 2, wherein the expression cassette comprises the coding sequence for an anti-Her2 immunoglobulin light chain having an IL-2 leader peptide consisting of the nucleotides 2745 to 3452 of SEQ ID NO: 17.

10. The recombinant nucleic acid molecule according to claim 2, wherein the linker is a furin-2A self-cleaving linker.

11. The recombinant nucleic acid molecule according to claim 2, wherein the regulatory sequences comprise an intron which is a chimeric intron.

12. The recombinant nucleic acid molecule according to claim 2, wherein the regulatory sequences comprise a SV40 polyadenylation (polyA) signal.

13. A plasmid comprising the recombinant nucleic acid molecule according to claim 2 and further comprising nucleic acid sequences upstream and downstream of the vector genome.

14. A packaging host cell in culture comprising (a) the recombinant nucleic acid molecule according to claim 2; (b) a recombinant nucleic acid molecule encoding an AAV rep protein; (c) a recombinant nucleic acid molecule encoding an AAV capsid protein; and (d) sufficient helper sequences to package the expression cassette into the AAV capsid.

15. The packaging host cell in culture according to claim 14, wherein the packaging host cell is a HEK293 cell.

16. The packaging host cell in culture according to claim 14, wherein the AAV capsid is AAV capsid which targets the central nervous system (CNS).

17. The packaging host cell in culture according to claim 14, wherein the AAV capsid is AAV9 capsid, AAVrh10 capsid, or AAVhu37 capsid.

18. A method for producing recombinant adeno-associated virus (rAAV) particles in a packaging host cell comprising culturing the packaging host cells of claim 14, wherein the host cell express rep and capsid proteins and package the expression cassette into assembled capsid to produce the rAAV particles.
